(19) United States
(12) Patent Application Publication
(10) Pub. No.: US 2010/0249497 A1
(43) Pub. Date: Sep. 30, 2010

(54) Surgical instrument

(76) Inventors: William J. Peine, Ashland, MA (US); Woojin Lee, Hopkinton, MA (US)

Correspondence Address: David M. Driscoll, Esq, 1201 Canton Avenue, Milton, MA, 02186, US

(21) Appl. No.: 12/584,988

(22) Filed: Sep. 15, 2009

Related U.S. Application Data
(60) Provisional application No. 61/211,410, filed on Mar. 30, 2009.

(51) Int. Cl. A61B 1/00

(52) U.S. Cl. 600/104

(57) ABSTRACT

A medical instrument that includes an instrument shaft having proximal and distal ends; a tool for performing a medical procedure; a control handle; a distal motion member for coupling the distal end of the instrument shaft to the tool; a proximal motion member for coupling the proximal end of the instrument shaft to the control handle; actuation means extending between the distal and proximal motion members for coupling motion of the proximal motion member to the distal motion member for controlling the positioning of the tool; a control tube through which the instrument shaft and tool extend; the control tube including, along the length thereof, a curved section; the curved section of the control tube, upon rotation thereof, providing an additional degree of freedom by displacing the tool out of a plane defined by the curved section of the control tube.

### RELATED APPLICATION

Priority for this application is hereby claimed under 35 U.S.C. 119(e) to commonly owned and co-pending U.S. Provisional Patent Application No. 61/211,410 which was filed on Mar. 30, 2009. The content of all of the aforementioned application is hereby incorporated by reference herein in its entirety.

### TECHNICAL FIELD

The present invention relates in general to medical instruments, and more particularly to manually-operated surgical instruments that are intended for use in minimally invasive surgery or other forms of surgical or medical procedures or techniques. The instrument described herein is primarily for a laparoscopic procedure, however, it is to be understood that the instrument of the present invention can be used for a wide variety of other procedures, including intraluminal procedures.

### BACKGROUND OF THE INVENTION

Endoscopic and laparoscopic instruments currently available in the market are extremely difficult to learn to operate and use, mainly due to a lack of dexterity in their use. For instance, when using a typical laparoscopic instrument during surgery, the orientation of the tool of the instrument is solely dictated by the location of the target and the incision. These instruments generally function with a fulcrum effect using the patient's own incision area as the fulcrum. As a result, common tasks such as suturing, knotting and fine dissection have become challenging to master. Various laparoscopic instruments have been developed over the years to overcome this deficiency, usually by providing an extra articulation often controlled by a separately disposed control member for added control. However, even so these instruments still do not provide enough dexterity to allow the surgeon to perform common tasks such as suturing, particularly at any arbitrarily selected orientation.

The goal of minimally invasive surgery (MIS) is to manipulate tissues within the human body while minimizing damage to the surrounding healthy organs. Laparoscopy, for example, uses endoscopic cameras and long slender instruments to perform surgery through a few small (1-2 cm) skin incisions. This provides many benefits to patients over traditional open incision techniques, including fewer infections, less pain, shorter hospital stays, faster recovery times, and less scaring. These advantages have allowed surgeons to apply MIS to procedures in every surgical specialty. During the 1990's, the growth rate of MIS was tremendous; however, in the last few years the application to new procedures has largely stalled due to limitations in visualization, access, and control. It is a general belief among surgeons that a new wave of technology is needed in order for MIS to reach the next level. Smaller cameras and instruments that can flexibly navigate around organs with added dexterity will allow them to perform surgery not possible today.

Prior laparoscopic and endoscopic instruments were a simple adaptation of tools used for open incision surgery. They are similar in mechanical construction with the addition of a long, 2˜10 mm diameter shaft between the handle and end effectors. They lack the dexterity of open incision surgery due to the “fulcrum effect”. Since the tools pivot about the incision, they are generally limited to 5 Degrees-of-Freedom (DOF): pivoting up/down, pivoting left/right, sliding in/out, rotating about the shaft axis, and actuation of the jaws. In contrast, open incision surgery allows full dexterity (7 DOF) due to the surgeon's wrist, with additional DOF from their elbow and shoulder used to avoid obstacles and optimize access to the tissue. Further complicating MIS, the surgeon views the operative site on a monitor located outside the sterile field. This displacement between eyes and hands combined with the reversal of motions caused from the fulcrum effect makes these techniques difficult to learn and master. It takes the skills of an experienced surgeon to consistently perform advanced MIS at a high level.

Surgery now in virtually every surgical discipline is moving toward making MIS more minimal. This means using smaller and fewer incisions, or most ideally, no incisions. The art has already made the transition from open to endoscopic surgery; now surgeons are pioneering surgical techniques that use the patient's natural orifices as entry points into the body. These approaches further reduce pain and recovery times and, in many cases, produce no visible scars.

One fairly new technique is referred to as single port access surgery (or SPA). This is a type of laparoscopy where all the instruments and laparoscope enter the abdominal cavity through one incision. Most of these procedures use the umbilicus for the entry port location because it heals quickly, does not have significant muscle groups below it, and hides any scaring well. Since the instruments enter the body at one location and operate in the same area of the abdomen, there is some limitation on the control of straight shaft instruments. Because only a single port is used there is a tendency, when using multiple instruments, to have one instrument interfere with the positioning of another instrument.

An object of the present invention is to provide an improved medical instrument that provides greater tool control and improved dexterity.

Another object of the present invention is to provide an improved surgical instrument that allows free non-interference control particularly when using multiple instruments.

A further object of the present invention is to provide an improved medical instrument that is characterized by the ability to lock the instrument in a pre-selected particular position.

Still another object of the present invention is to provide a locking feature that is an important adjunct to the other controls of the instrument enabling the surgeon to lock the instrument once in the desired position. This makes it easier for the surgeon to thereafter perform surgical procedures without having to, at the same time, hold the instrument in a particular bent configuration.

### SUMMARY OF THE INVENTION

To accomplish the foregoing and other objects, features and advantages of the present invention there is provided a medical instrument comprising: an instrument shaft having proximal and distal ends; a tool for performing a medical procedure; a control handle; a distal motion member for coupling the distal end of the instrument shaft to the tool; a proximal motion member for coupling the proximal end of the instrument shaft to the control handle; actuation means extending between the distal and proximal motion members for coupling motion of the proximal motion member to the distal motion member for controlling the positioning of the tool; a control tube through which the instrument shaft and tool extend; the control tube including, along the length thereof, a curved section; the curved section of the control tube, upon rotation thereof, providing an additional degree of freedom by displacing the tool out of a plane defined by the curved section of the control tube.

Other aspects of the present invention include at least a portion of the length of the instrument shaft is flexible so as to enable the instrument shaft to pass through the curved section of the control tube; a ball member supported about the proximal motion member, the control tube having a distal end and a proximal end with the proximal end of the control tube fixedly attached to the ball member; the control tube is rigid and includes a straight section proximal to and contiguous with the curved section; the instrument shaft extends through the curved control tube so that the distal motion member and tool extend beyond the distal end of the curved control tube; a rotation knob at the control handle for rotating the instrument shaft and end effector about a longitudinal distal axis; both of the motion members are bendable members; a ball member supported about the proximal bendable member, the control tube having a distal end and a proximal end with the proximal end of the control tube fixedly attached to the ball member, and a locking mechanism disposed about the ball member; the locking mechanism includes a cinch ring that can be expanded and contracted; the control tube is rigid and includes a straight section proximal to and contiguous with the curved section, the straight and curved sections defining the plane.

In accordance with another version of the present invention there is also provided a medical instrument comprising: an instrument shaft having proximal and distal ends; a tool for performing a medical procedure; a control handle; a distal motion member for coupling the distal end of the instrument shaft to the tool; a proximal motion member for coupling the proximal end of the instrument shaft to the control handle; actuation means extending between the distal and proximal motion members for coupling motion of the proximal motion member to the distal motion member for controlling the positioning of the tool; a control tube through which the instrument shaft and tool extend; the control tube including, along the length thereof, a curved section; and a guide block having a slot therein for receiving the instrument shaft and control tube; the guide block disposed proximally of an anatomic port.

Other aspects of the present invention include the curved section of the control tube, upon rotation thereof, providing an additional degree of freedom by displacing the tool out of a plane defined by the curved section of the control tube; the control tube has at least three curved sections disposed therealong; two of the curved sections are proximal to the guide block and one of the curved sections is distal to the guide block; including a pair of instruments and wherein the guide block has a corresponding pair of slots for receiving respective instrument shafts.

In still another version of the present invention there is provided a medical instrument comprising: an instrument shaft having proximal and distal ends; a tool for performing a medical procedure; a control handle; a distal motion member for coupling the distal end of the instrument shaft to the tool; a proximal motion member for coupling the proximal end of the instrument shaft to the control handle; actuation means extending between the distal and proximal motion members for coupling motion of the proximal motion member to the distal motion member for controlling the positioning of the tool; a control tube through which the instrument shaft and tool extend; the control tube including, along the length thereof, a at least one curved section; and an over tube having a passage therein for receiving the instrument shaft and control tube; the over tube disposed proximally of an anatomic port.

Still further aspects of the present invention include at least one flexible articulation section along the length of the control tube; the curved section of the control tube is distal of the over tube and is rigid; including a flexible articulation section on either side of the over tube and connected by cabling therebetween; the proximal motion member comprises a cable drive mechanism; the cable drive mechanism includes at least one motor, at least one pair of cables and a corresponding pair of followers driven by the motor; including a threaded shaft for supporting the followers, driven from the motor and having opposed threads to drive the followers in opposite directions in controlling the cables; including four cables and two motors mounted at the handle.

### BRIEF DESCRIPTION OF THE DRAWINGS

It should be understood that the drawings are provided for the purpose of illustration only and are not intended to define the limits of the disclosure. The foregoing and other objects and advantages of the embodiments described herein will become apparent with reference to the following detailed description when taken in conjunction with the accompanying drawings in which:

FIG. 1 is a perspective view of a first embodiment of the surgical tool with a single bend in the curved tube;

FIG. 2 is a fragmentary cross-sectional view of the instrument of FIG. 1 as taken along line 2-2 of FIG. 1, and additionally illustrating the handle and end effector bent in relation to the instrument shaft;

FIG. 3 is a fragmentary cross-sectional view of the distal end of the instrument as taken along line 3-3 of FIG. 2;

FIG. 4 is a fragmentary cross-sectional view of the proximal end of the instrument as taken along line 4-4 of FIG. 2;

FIG. 5 is a cross-sectional view taken along line 5-5 of FIG. 3;

FIG. 6 is a cross-sectional view taken along line 6-6 of FIG. 3;

FIG. 7 is a cross-sectional view taken along line 7-7 of FIG. 4;

FIG. 8 is a cross-sectional view taken along line 8-8 of FIG. 2;

FIG. 9 is a fragmentary enlarged cross-sectional view of the area encircled by arrow-9-9 of FIG. 8 but showing an alternate embodiment of the locking means;

FIG. 10 is a cross-sectional view similar to that shown in FIG. 9 but showing the angle locking means engaged;

FIG. 11 is a cross-sectional view similar to that shown in FIG. 8 but showing an alternate embodiment of locking means and the cinch ring in an alternate unlatched position;

FIG. 12 is a cross-sectional view as taken along line 12-12 of FIG. 11;

FIGS. 12A-12E are a series of schematic fragmentary perspective views illustrating the oscillating motion of the ball member;

FIG. 13 is a cross-sectional view similar to that shown in FIG. 8 but showing an alternate embodiment of control tube angle locking means;

FIG. 14 is a cross-sectional view as taken along line 14-14 of FIG. 13;

FIG. 15 is a cross-sectional view similar to that shown in FIG. 13 but showing the angle locking mechanism in a locked position;

FIG. 16 is a cross-sectional view as taken along line 16-16 of FIG. 15;

FIGS. 17A-17E are diagrammatic perspective views showing the different ways of manipulating the instruments during a surgical procedure;

FIG. 18 is a perspective view of an alternate embodiment of instrument in use;

FIG. 19 is a fragmentary cross-sectional view of the instrument shaft and control tube taken along line 19-19 of FIG. 18;

FIGS. 20A-20C, 21A-21C and 22A-22C are respective diagrammatic plan, rear and side views showing different ways of manipulating the instruments shown in FIG. 18 during a surgical procedure;

FIG. 23 is a perspective view of an alternate embodiment of instrument in use;

FIG. 24 is a cross-sectional view of the articulation sections and mid portion of the control tube as taken along line 24-24 of FIG. 23;

FIGS. 25 and 26 are cross-sectional views as taken along lines 25-25 and 26-26 of FIG. 24;

FIG. 27 is a cross-sectional view similar to that shown in FIG. 24 but showing the articulation sections in a bent position;

FIG. 28 is a schematic view of FIG. 27;

FIG. 29 is a schematic view similar to that shown in FIG. 27 but with an alternate embodiment of cabling means;

FIG. 30 is a somewhat schematic cross-sectional view of the cable drive mechanism for the end effector;

FIG. 30A is a cross-sectional view as taken along line 30A-30A of FIG. 30;

FIGS. 31-33 are diagrammatic cross-sectional side views showing operation of the cable drive mechanism;

FIG. 34 is an enlarged fragmentary cross-sectional view of an alternate embodiment of cable drive mechanism;

FIG. 34A is a cross-sectional view as taken along line 34A-34A of FIG. 34;

FIG. 35 is a schematic plan view of an alternate embodiment of the instrument; and

FIG. 35A is a cross-sectional view as taken along line 35A-35A of FIG. 35.

### DETAILED DESCRIPTION

The instrument of the present invention may be used to perform minimally invasive procedures. “Minimally invasive procedure,” refers herein to a surgical procedure in which a surgeon operates through a small cut or incision, the small incision being used to access the operative site. In one embodiment, the incision length ranges from 1 mm to 20 mm in diameter, preferably from 5 mm to 10 mm in diameter. This procedure contrasts those procedures requiring a large cut to access the operative site. Thus, the instrument is preferably used for insertion through such small incisions and/or through a natural body lumen or cavity, so as to locate the instrument at an internal target site for a particular surgical or medical procedure. The introduction of the surgical instrument into the anatomy may also be by percutaneous or surgical access to a lumen, vessel or cavity, or by introduction through a natural orifice in the anatomy.

In addition to use in a laparoscopic procedure, the instrument of the present invention may be used in a variety of other medical or surgical procedures including, but not limited to, colonoscopic, upper GI, arthroscopic, sinus, thorasic, prostate, transvaginal, orthopedic and cardiac procedures. Depending upon the particular procedure, the instrument shaft may be rigid, semi-rigid or flexible.

Although reference is made herein to a “surgical instrument,” it is contemplated that the principles of this invention also apply to other medical instruments, not necessarily for surgery, and including, but not limited to, such other implements as catheters, as well as diagnostic and therapeutic instruments and implements.

There are a number of important features embodied in the instrument of the present invention. One significant feature is the ability to add another degree of freedom to the instrument by having the instrument shaft pass through a rigid curved control tube. The additional DOF is obtained by rotating the instrument handle to orbit the distal tool in and out of a plane that is initially defined by the curved tube. The instrument itself adds the further degrees of freedom via the bendable members and rotation knob, as well as by using the “fulcrum effect”.

A further feature embodied in the instrument of the present invention relates to providing a locking mechanism that is constructed using a ball and socket arrangement disposed about the proximal motion member that follows the bending action and in which an annular cinch ring is used to retain the ball and socket arrangement in a fixed particular position, and thus also maintain the proximal and distal bendable members in a particular bent condition, or in other words locked in that position. The cinch ring includes a locking lever that is conveniently located adjacent to the instrument handle and that is easily manipulated to lock and unlock the cinch ring and, in turn, the position of the end effector. The cinch ring is also preferably rotatable to that the locking lever can be positioned conveniently or can be switched (rotated) between left and right handed users. This lock control allows the surgeon one less degree of freedom to concentrate on when performing certain tasks. By locking the bendable sections at a particular position, this enables the surgeon to be more hands-free for controlling other degrees of freedom of the instrument such as manipulation of the rotation knob to, in turn, control the orientation of the end effector.

Another feature of the present invention relates to the manner in which the bending is carried out. In the past, relatively small diameter flexible cables have been used to control bending between the proximal and distal bendable members. However, this has caused a somewhat uneven control in that there was only a “pulling” action by one cable while the opposite cable relaxed. The present instrument uses a more rigid cable arrangement so that the bending occurs with both a “pulling” action as well as an opposed “pushing” action. To do this the cables are of larger relative diameter and somewhat rigid, but still have to have sufficient flexibility so that they can readily bend. Also, the cables are preferably constrained along their length so as to prevent cable deflection or buckling, particularly during the “pushing” phase of a cable.

Still another feature is the pistol grip arrangement and the control lever which has an end gimbal construction that provides for a more precise control of the actuation lever and the corresponding actuation of the end effector. Also the control lever in accordance with the present instrument is provided with a means to control the attitude of the control lever to compensate for different configurations of hands, particularly to compensate for the different length fingers of a user.

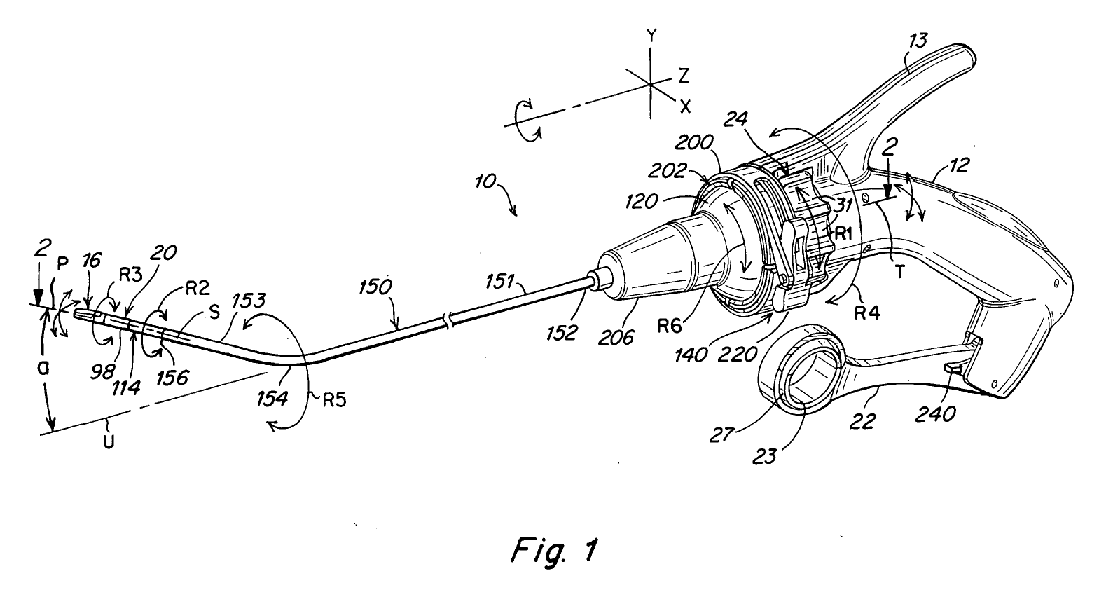

FIG. 1 is a perspective view of one embodiment of the surgical instrument 10 of the present invention. In this surgical instrument both the tool and handle motion members or bendable members are capable of bending in any direction. They are interconnected via cables 100 (preferably four cables) in such a way that a bending action at the proximal member provides a related bending at the distal member. The proximal bending is controlled by a motion or deflection of the control handle by a user of the instrument. In other words the surgeon grasps the handle and once the instrument is in position any motion (deflection) at the handle immediately controls the proximal bendable member which, in turn, via cabling controls a corresponding bending or deflection at the distal bendable member. This action, in turn, controls the positioning of the distal tool. This action is coupled with the aforementioned curved tube control.

The proximal member is preferably generally larger than the distal member so as to provide enhanced ergonomic control. In the illustrated embodiment the ratio of proximal to distal bendable member diameters may be on the order of three to one. In one version in accordance with the invention there may be provided a bending action in which the distal bendable member bends in the same direction as the proximal bendable member. In an alternate embodiment the bendable, turnable or flexible members may be arranged to bend in opposite directions by rotating the actuation cables through 180 degrees, or could be controlled to bend in virtually any other direction depending upon the relationship between the distal and proximal support points for the cables.

As has been noted, the amount of bending motion produced at the distal bending member is determined by the dimension of the proximal bendable member in comparison to that of the distal bendable member. In the embodiment described, the proximal bendable member is generally larger than the distal bendable member, and as a result, the magnitude of the motion produced at the distal bendable member is greater than the magnitude of the motion at the proximal bendable member. The proximal bendable member can be bent in any direction (about 360 degrees) controlling the distal bendable member to bend in either the same or an opposite direction, but in the same plane at the same time. Also, as depicted in FIG. 1, the surgeon is able to bend and roll the instrument's tool about its longitudinal axis to any orientation simply by rolling the axial rotation knob 24 about the direction of the rotation arrow R1.

In this description reference is made to bendable members. These members may also be referred to as turnable members, bendable sections or flexible members. In the descriptions set out herein, terms such as “bendable section,” “bendable segment,” “bendable member,” or “turnable member” refer to an element of the instrument that is controllably bendable in comparison to an element that is pivoted at a joint. The term “movable member” is considered as generic to bendable sections and joints. The bendable elements of the present invention enable the fabrication of an instrument that can bend in any direction without any singularity and that is further characterized by a ready capability to bend in any direction, all preferably with a single unitary or uni-body structure. A definition of a “unitary” or “uni-body” structure is—a structure that is constructed only of a single integral member and not one that is formed of multiple assembled or mated components—.

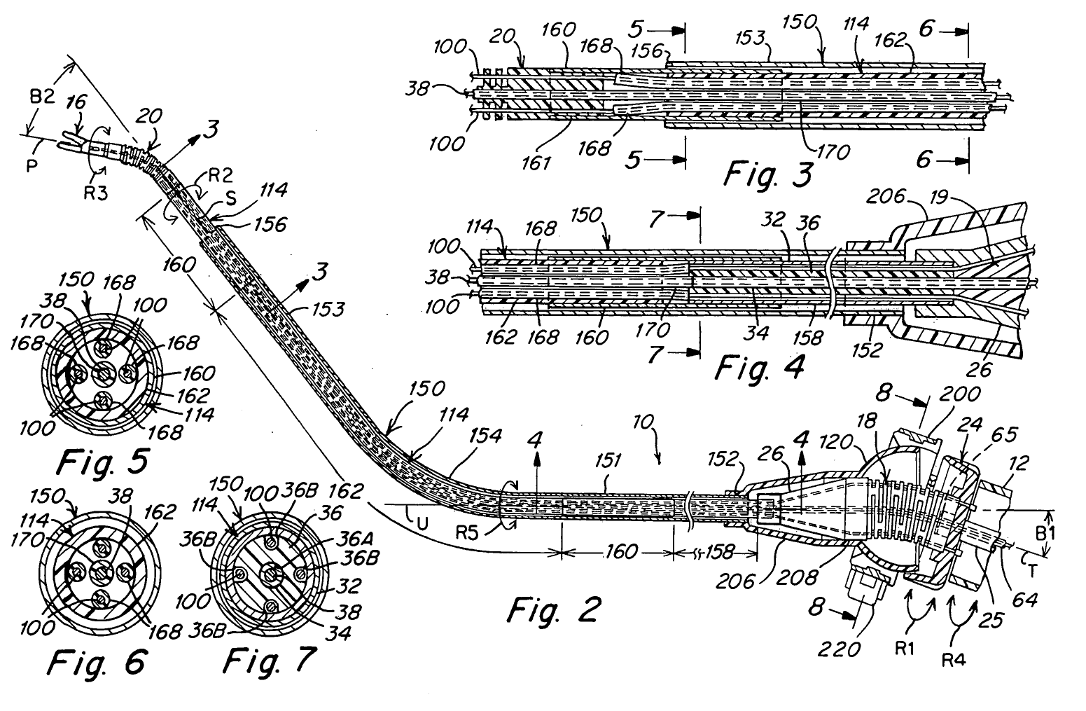

A definition of these bendable members is—an instrument element, formed either as a controlling means or a controlled means, and that is capable of being constrained by tension or compression forces to deviate from a straight line to a curved configuration without any sharp breaks or angularity—. Bendable members may be in the form of unitary structures, such as shown herein in FIGS. 2 and 3, may be constructed of engageable discs, or the like, may include bellows arrangements or may comprise a movable ring assembly. For other forms of bendable members refer to co-pending application Ser. No. 11/505,003 filed on Aug. 16, 2006 and Ser. No. 11/523,103 filed on Sep. 19, 2006, both of which are hereby incorporated by reference herein in their entirety.

FIG. 1 is a perspective view of a first embodiment of a surgical instrument in which the instrument part itself may be of the type described in U.S. Ser. Nos. 11/528,134 filed on Sep. 27, 2006 and 11/649,352 filed on Jan. 2, 2007, both of which are hereby incorporated by reference herein in their entirety.

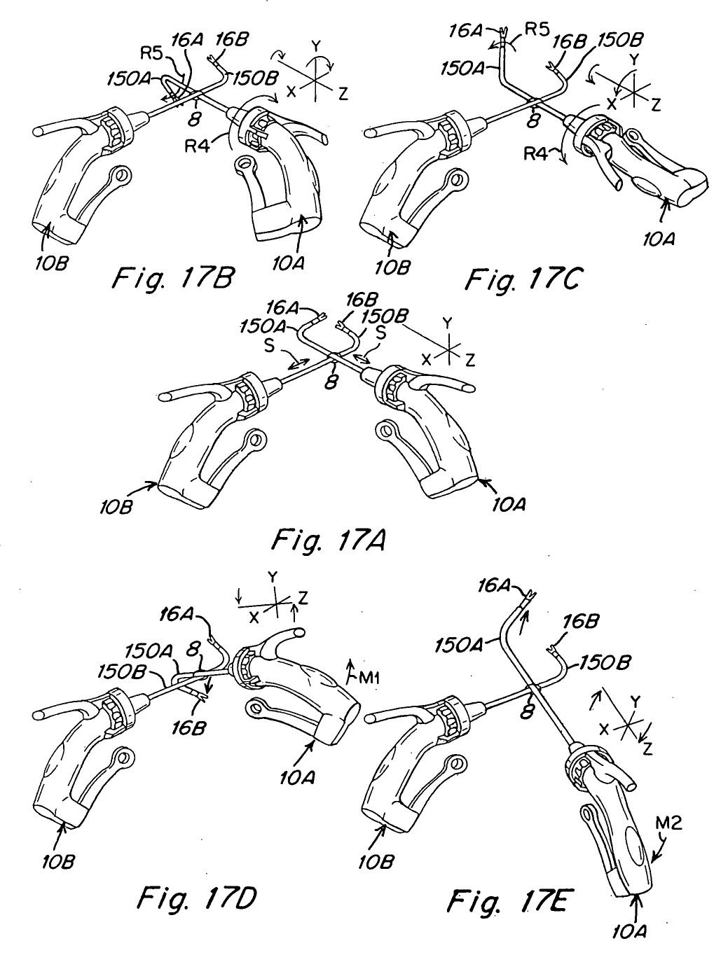

In FIG. 1 the instrument 10 has been inserted into the curved or bent control tube 150. The control tube 150 is rigid preferably along its entire length. In FIG. 1 the Instrument is shown in a neutral position with zero angle between the longitudinal instrument handle axis T and proximal shaft portion longitudinal axis U, and with resulting zero angle between the distal shaft portion longitudinal axis S and the distal tip tool axis P. In that position both of the bendable members 18, 20 are in a straight or non-bent position. The primary difference between the instrument of the present invention and an instrument as shown in Ser. No. 11/649,352 is the addition of the bent or curved control tube 150 which is rigidly attached at its proximal end 152 to the neck portion 206 of ball 120. The tube 150 includes a straight section 151 at its proximal end, a distal end section 153 which is also shown as straight and a bend 154 therebetween that is located approximately three fourths of the distance to the distal end 156, as is shown in, for example, FIG. 17A.

In FIG. 1 the bend 154 is illustrated as at an angle “a” towards the right side of the tool in the direction of axis X (negative X axis). Alternately, the bend may be oriented towards the left side of the tool in the direction of the X axis by rotating the ball 120 and control tube 150 by 180 degrees. The left-right orientation of two tools as may be used in laparascopic surgery is illustrated in FIGS. 17A-17E. The instruments of the present invention including the curved tubes are instrumental in the proper triangulation of the instrument tips while allowing the separation of handles outside of the patient during surgery in order to avoid collisions. FIGS. 17A-17E represent clear examples of how the tips of the instruments are disposed in relatively close proximity for the purpose of performing a surgery technique, while the handles are spaced apart so as to provide easy and unobstructed control of each instrument without interference between the instruments.

The instrument of the present invention may be used for laparoscopic surgery through the abdominal wall. For this purpose there is typically provided an insertion site at which there is disposed a cannula or trocar. The shaft 114 of the instrument 10, as well as the curved tube 150 is adapted to pass through the cannula or trocar so as to dispose the distal end of the instrument at the operative site. The end effector 16 as depicted in FIG. 1 may be considered as disposed at an operative site with the cannula or trocar at the incision point in the skin. The embodiment of the instrument shown in FIG. 1 may be used with a sheath at the distal end thereof to keep bodily fluids from entering the distal bending member 20.

A rotation motion can be carried out with the instrument of the present invention. This can occur by virtue of the rotation of the rotation knob 24 relative to the handle 12 about axis T (refer to FIG. 1). This is represented in FIG. 1 by the rotation arrow R1. When the rotation knob 24 is rotated, in either direction, this causes a corresponding rotation of the instrument shaft 114. This is depicted in FIGS. 1 and 2 by the rotational arrow R2. This same motion also causes a rotation of the distal bendable member and end effector 16 about an axis that corresponds to the instrument tip, depicted in FIGS. 1 and 2 as about the longitudinal tip or tool axis P (arrow R3). This distal rotation is relative to the control tube 150.

Any rotation of the rotation knob 24 while the instrument is locked (or unlocked) maintains the instrument tip at the same angular position, but rotates the orientation of the tip (tool). For a further explanation of the tip rotational feature refer to co-pending application Ser. No. 11/302,654, filed on Dec. 14, 2005, particularly FIGS. 25-28, which is hereby incorporated by reference in its entirety.

The handle 12, via proximal bendable member 18, may be tilted at an angle to the instrument shaft longitudinal center axis. This tilting, deflecting or bending may be considered as in the plane of the paper. By means of the cabling this action causes a corresponding bend at the distal bendable member 20 to a position wherein the tip is directed along an axis and at a corresponding angle to the instrument shaft longitudinal center axis. The bending at the proximal bendable member 18 is controlled by the surgeon from the handle 12 by manipulating the handle in essentially any direction. This manipulation directly controls the bending at the proximal bendable member. Refer to FIG. 2 in which there is shown the axis U corresponding to the instrument shaft longitudinal axis. Refer also to the proximal bend angle B1 between axes T and U, and the corresponding distal bend angle B2 between axes S and P.

Thus, the control at the handle is used to bend the instrument at the proximal motion member to, in turn, control the positioning of the distal motion member and tool. The “position” of the tool is determined primarily by this bending or motion action and may be considered as the coordinate location at the distal end of the distal motion member. Actually, one may consider a coordinate axis at both the proximal and distal motion members as well as at the instrument tip. This positioning is in three dimensions. Of course, the instrument positioning is also controlled to a certain degree by the ability of the surgeon to pivot the instrument at the incision point (port 8), as well as rotation of curved control tube to displace the distal tool. The “orientation” of the tool, on the other hand, relates to the rotational positioning of the tool, from the proximal rotation control member (rotation knob 24), about the illustrated distal tip or tool axis P.

In the drawings a set of jaws is depicted, however, other tools or devices may be readily adapted for use with the instrument of the present invention. These include, but are not limited to, cameras, detectors, optics, scope, fluid delivery devices, syringes, etc. The tool may include a variety of articulated tools such as: jaws, scissors, graspers, needle holders, micro dissectors, staple appliers, tackers, suction irrigation tools and clip appliers. In addition, the tool may include a non-articulated tool such as: a cutting blade, probe, irrigator, catheter or suction orifice.

The surgical instrument of FIG. 1 shows a preferred embodiment of a surgical instrument 10 according to the invention in use and may be inserted through a cannula at an insertion site through a patient's skin and depicted in the drawings as port 8. Many of the components shown herein, such as the instrument shaft 114, end effector 16, distal bending member 20, and proximal bending member 18 may be similar to and interact in the same manner as the instrument components described in the co-pending U.S. application Ser. No. 11/185,911 filed on Jul. 20, 2005 and hereby incorporated by reference herein in its entirety. Many other components shown herein, particularly at the handle end of the instrument may be similar to components described in the co-pending U.S. application Ser. No. 11/528,134 filed on Sep. 27, 2006 and hereby incorporated by reference herein in its entirety. Also incorporated by reference in their entirety are U.S. application Ser. No. 10/822,081 filed on Apr. 12, 2004; U.S. application Ser. No. 11/242,642 filed on Oct. 3, 2005 and U.S. application Ser. No. 11/302,654 filed on Dec. 14, 2005, all commonly owned by the present assignee.

As illustrated in FIGS. 2-4, the control between the proximal bendable member 18 and distal bendable member 20 is provided by means of the bend control cables 100. In the illustrated embodiment four such control cables 100 are provided in order to provide the desired all direction bending. However, in other embodiments of the present invention fewer or less numbers of bend control cables may be used. The bend control cables 100 extend through the instrument shaft 114 and through the proximal and distal bendable members. The bend control cables 100 are preferably constrained along substantially their entire length so as to facilitate both “pushing” and “pulling” action. The cables 100 are also preferably constrained as they pass over the conical cable guide portion 19 of the proximal bendable member, and through the proximal bendable member. Refer, for example, to FIG. 4.

The locking means of the present instrument interacts with the ball and socket arrangement to lock and unlock the positioning of the cables which in turn control the angle of the proximal bending member and thus the angle of the distal bendable member and end effector. This lock control allows the surgeon one less degree of freedom to concentrate on when performing certain tasks. By locking the bendable sections at a particular position, this enables the surgeon to be more hands-free for controlling other degrees of freedom of the instrument such as manipulation of the rotation knob 24 and, in turn, orientation of the end effector.

The instrument shown in FIG. 1 is of a pistol grip type. However, the principles of the present invention may also apply to other forms of handles such as a straight in-line handle. In FIG. 1 there is shown a jaw clamping means that is comprised mainly of the lever 22 which has a single finger hole for controlling the lever and also may include a related release function controlled directly by the lever 22 rather than a separate release button. The release function is used to release the actuated or closed tool.

In the instrument that is illustrated the handle end of the instrument may be tipped in any direction as the proximal bendable member is constructed and arranged to enable full 360 degree bending. This movement of the handle relative to the instrument shaft bends the instrument at the proximal bendable member 18. This action, in turn, via the bend control cables 100, bends the distal bendable member in the same direction. As mentioned before, opposite direction bending can be used by rotating or twisting the control cables through 180 degrees from one end to the other end thereof.

In the embodiment described herein, the handle 12 is in the form of a pistol grip and includes a horn 13 to facilitate a comfortable interface between the action of the surgeon's hand and the instrument. The tool actuation lever 22 is shown in FIG. 1 pivotally attached at the base of the handle. The lever 22 actuates a slider (not shown) that controls a tool actuation cable 38 (FIGS. 3 and 4) that extends from the slider to the distal end of the instrument. The cable 38 controls the opening and closing of the jaws, and different positions of the lever control the force applied at the jaws.

The shape of the handle allows for a comfortable and substantially one-handed operation of the instrument as shown in FIG. 1. As shown in FIG. 1, the surgeon may grip the handle 12 between his palm and middle finger with the horn 13 nestled in the crook between his thumb and forefinger. This frees up and positions the forefinger and thumb to rotate the rotation knob 24 using the finger indentions 31 that are disposed on the peripheral surface of the rotation knob, as depicted in FIG. 1. In both locked and unlocked positions of the instrument the rotation knob is capable of controlled rotation to control axial rotation at the tip of the instrument about the distal tool tip axis P, as represented by the rotation arrow R3 in FIGS. 1 and 2.

In the disclosed embodiment there is provided at the tool closing lever 22 a fingertip engaging recess 23 in a gimbaled ball 27. The free end of the lever 22 supports the gimbaled ball 27 which has the through hole or recess 23 which receives one of the fingers of the user. The ball 27 is free to at least partially rotate in three dimensions in the lever end. The surgeon may grip the handle between the palm, ring and pinky fingers with the horn 13 nestled in the crook between his thumb and forefinger and operate the rotation knob 24 as previously described. The surgeon may then operate the jaw clamping lever 22 with the forefinger or middle finger.

The gimbaled ball 27 is in the form of a ball in a socket, in which the ball 27 is free to be rotated in the socket, and in which the socket is defined in the lever free end. In this embodiment, rather than having the hole or recess 23 go completely through the ball there is preferably provided a blind hole in the ball. The ball is free to rotate in the lever end and thus the ball can also be rotated to alternate positions, such as through 180 degrees, corresponding to either a right-handed or left-handed user. The blind hole (in comparison to a through hole) enables the user to have a firmer grip of the lever and thus enhanced control of the lever action.

The jaw clamping lever 22 is also adjustable for left and right handed operation as well as a range of other adjustments. Refer to Ser. No. 11/649,352 for further details of this control. This control is basically accomplished by means of the cam lever 240 that adjusts the attitude of the clamping lever 22 relative to a center line or center plane of the handle. This adjustment can be made based on whether the user is right handed or left handed, or can be made on the basis of some other characteristic of the hand of the user such as finger length.

The locking mechanism or angle locking means 140 of the instrument includes a ball and socket arrangement that is basically disposed over the proximal bendable member and that follows the bending at the proximal bendable member. The locking mechanism has locked and unlocked positions, is disposed about the proximal movable or bendable member and is manually controlled so as to fix the position of the proximal movable member relative to the handle in the locked position thereof. The locking mechanism comprises a ball member and a compressible hub that defines a socket member. In the disclosed embodiment the hub is a split hub and the locking mechanism further includes a cinch ring disposed about the split hub and a locking lever mounted on the cinch ring for closing the cinch ring about the hub to lock the hub against the spherical ball member, and thus lock the bendable members in a particular relative position. The cinch ring interlocks with the hub but is able to rotate relative thereto when in the unlocked position. Again, reference is made to Ser. No. 11/649,352 for further details of this feature.

The “ball” part is basically formed by the ball member 120, while the “socket” part is basically formed by an extension of the handle, namely the split hub 202. The locking mechanism locks the proximal bendable member in a desired position and by doing that also locks the position of the distal bendable member and tool. The proximal bending member 18, although it is enclosed the ball and socket arrangement, still allows the instrument shaft and the proximal bending member 18, along with the cabling 100, to rotate freely while also allowing the axis of the instrument shaft to be angled relative to the axis of the handle in a free, or alternately, locked mode.

The ball member 120 is gimbaled in a split hub 202 that is comprised of four quadrants 202A-202D that can be clamped against the spherical surface 204 of the ball member 120 by means of the cinch ring 200. Refer to FIG. 8. The split hub 202 may be supported at the distal end of the handle by means of a set of struts. The ball member 120 has a neck portion 206 that provides support for the distal end of the proximal bendable member 18. In this regard a bearing surface 208 is provided, as illustrated in FIG. 2, between the proximal end of the neck 206 and the adaptor 26. This enables the proximal bendable member, along with the adaptor 26 to be free to rotate relative to the ball member 120.

FIGS. 1 and 8 illustrate the cinch ring 200. The cinch ring is an annular member that may have an internal ridge or spline that is adapted to mate with a channel or groove in the outer surface of the split hub 202. When used this combination of a channel and ridge limits the annular cinch member to just rotation about the hub 202. FIG. 8 also shows each of the portions 200A-200B of the split hub that may connect to the instrument handle via respective struts. When the cinch ring 200 is closed this, in turn, closes the slotted hub and essentially compresses the socket (hub 202) against the spherical surface 204 of the ball member 120. The locking of the ball member thus fixes the position of the proximal bendable member, and, in turn, the position of the distal bendable member and tool via the angle locking means 140.

The cinch ring 200 is operated by means of an over-center locking lever 220 that is connected to ends 200A and 200B of the cinch ring 200 by means of the pins 224. The lock lever 220 may be in a locked position or a released or unlocked position. The end 200A of the cinch ring 200 is in the form of a detachable hook that snap fits over the pin 222 and sits in a slot of the lever 220 when the ring is locked. The other end 200B of the cinch ring 200 may be in the form of two bales that snap fit over pin 224 formed on the sides of the lever 220. The cinch ring 200 is free to rotate around the split hub 202 when lever 220 is released by means of a spline that rides in a groove in the circumference of the split hub 202. This allows for left or right handed operation of the instrument.

When the locking lever 220 is moved to its locked position this compresses the cinch ring 200 closing the hub against the spherical outer surface 204 of the ball member 120. This locks the handle against the ball member 120 holding the ball member in whatever position it is in when the locking occurs. By holding the ball member in a fixed position this, likewise, holds the proximal bendable member in a particular position and fixed in that position. This, in turn, maintains the distal bendable member and tool at a fixed position, but the instrument orientation can be controlled via the control of the rotation knob which controls the orientation of the instrument tip by enabling rotation of the distal bendable member and tool about the tip axis P (see FIG. 3). Also, this rotational orientation is possible whether the locking mechanism is activated or not. In other words the tip of the instrument can be rotated about axis P in both the locked state and unlocked state of the angle locking means 140.

To adjust the orientation of the curved control tube 150, the release/lock lever 220 of the locking means 140 can be flipped to release it from its' over center position as is illustrated in FIG. 8. In this released position the cinch ring 200 is expanded and releases the segments 202A-202D of the split hub 202 to expand and release the spherical surface 204 of the ball 120 so that the ball, along with control tube 150, can be rotated for left or right hand use or for other adjustments. The horn 13 can be used as a reference point relative to the users hand so as to angle the control tube 150 virtually anywhere within the range of motion allowed by the ball and socket. In alternate embodiments discussed below the range of motion may be limited.

As indicated previously, by rotating the rotation knob 24 about axis T of the instrument, this results in a rotation of the entire length of the instrument shaft 114. As illustrated in FIG. 2, this includes a rotation R2 at the distal end of the instrument shaft (actually all along the shaft axis), as well as a rotation of the distal bending member 20 about axis S. This action also includes a rotation shown by rotation arrow R3 of the end effector 16 about the distal tip longitudinal axis P. This orientation of the tip of the instrument occurs regardless of the position of the curved control tube. However, with the control tube now added to the instrument a further degree of freedom of control is possible. With the rigid bent control tube 150 being fixedly attached to the ball member 120, when the handle 12 of the instrument is rotated about axis T, then this causes an orbiting effect regarding the positioning of the end effector or tool 16 relative to the instrument handle. This is illustrated in FIGS. 1 and 2 by the rotation arrow R5. In essence the rotation R4 causes the distal part of the instrument, particularly the tool 16 to raise or lower, as is illustrated in FIGS. 17B and 17C. In FIGS. 17B and 17C also refer to the arrows R4 and R5 and their direction of rotation to orient the tool. This “orbiting effect” is enabled by the use of a rigid curved tube that upon rotation thereof moves the distal part of the tube out of its initial plane thus orbiting the distal end of the control tube and any instrument mounted therein.

Moreover, in addition to controlling the curved tube 150 by rotating the handle, the position of the bent control tube 150 can also be adjusted by releasing the angle locking means 140. Once the locking means 140 is released by disengaging the cinch ring 200, then the ball member 120 is free to rotate in the direction of the rotational arrow R6, as illustrated in FIG. 1. As the control tube 150 is secured to the ball member 120, any rotation of the ball member 120 causes a like rotation of the control tube in the aforementioned orbiting manner. The control tube 150 can be thought of as having an initial position that defines an initial plane defined by the control tube itself. Upon rotation of the control tube, then the distal end of the control tube moves out of the initial plane, either upwardly or downwardly depending upon the direction of rotation.

Thus, the handle can be manipulated in a number of different ways including control of the control tube as just discussed, the bending action between proximal and distal bendable members and the ability of the surgeon to pivot the instrument at a fulcrum defined at the incision port 8. For the bending action, as mentioned before, when the handle 12 of the instrument is bent at angle B1 between the axis T of the handle and the axis U of the proximal end of the instrument shaft, the end effector 16 axis P is bent at an angle B2 to the axis S of the distal end 156 of the control tube 150, as illustrated in FIGS. 1 and 2. The bend in the control tube may be in a preferred range of 15 degrees to 75 degrees with a radius at the bend in the range of 0.5 inches to 3.0 inches. In order for the instrument shaft 114 to be able to rotate within the bent portion 154 of the control tube 150, a flexible section 162 has been added to the instrument shaft 114, as illustrated in FIGS. 2 and 3.

Rotation knob 24 and hub 25 are free to rotate about center wire conduit 64, restrained by the e-ring 65. The proximal bendable member 18 is seated in the rotation knob 24 and the conical end portion 19 is seated in the adapter 26 which is also free to rotate within neck 206 of the ball member 120 at bearing interface surface 208. A short rigid section 158 of the instrument shaft 114 is attached to the adapter 26, as shown in FIGS. 2 and 4, and is free to rotate within the proximal straight section 151 of the control tube 150. The rigid section 158 is made up of outer shaft tube 32 and shaft filler 36 with a lumen 36A (FIG. 7) for the inner shaft tube 34 and cable 38, as well as four grooves 36B (FIG. 7) for accommodating the cables 100. The rigid section 158 is attached to the flexible section 162 by a connector 160 that is preferably a short piece of stainless steel tubing about 2 inches long that is force fit or otherwise bonded to the flexible plastic tubing 162, as illustrated in FIG. 4. Because the flexible tubing 162 is hollow, PEEK tubes 168, 170 may be used to stiffen the push-pull cables 100, 38 respectively. Alternately, the flexible section 162 may be an extruded plastic with inner lumens to support the cables without having to use PEEK tubes. At the distal end, the flexible section 162 is connected by cylindrical connector 160 to a reduced neck portion 161 of the distal bendable member 20 which is articulated by cables 100. See the cross-sectional view of FIG. 3 that shows the cables 100 extending into the distal bendable member terminating at a distal end thereof, as well as the tool control cable 38. A sheath 98 may be used as illustrated in FIG. 1 to prevent bodily fluids from entering the distal end of the instrument, such as at openings that receive the bend control cables 100.

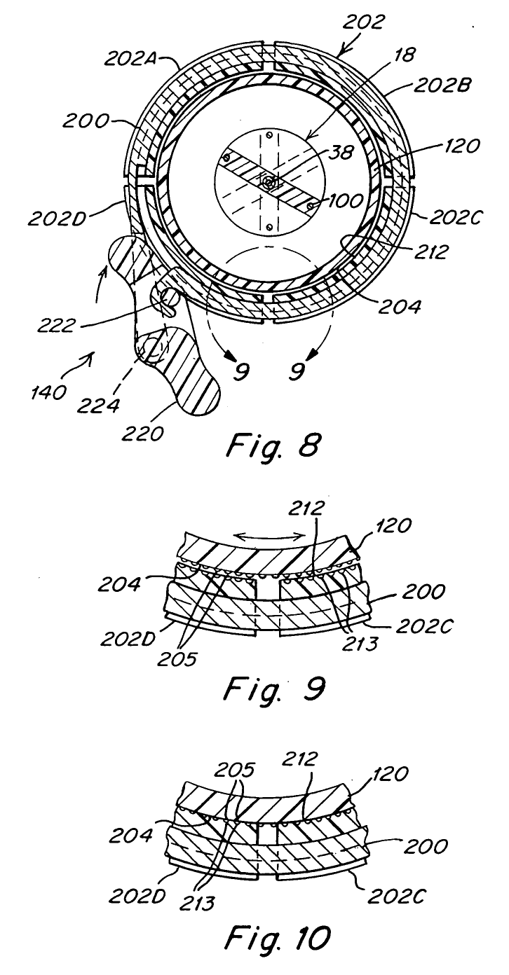

FIG. 8 is a cross-sectional view of the angle locking means 140 taken along line 8-8 of FIG. 2 and shows the release/lock lever 220 in a released position. In that position the cinch ring 200 expand enough to let the split hub segments 202A-202D release the surface 204 of the ball 120. The control tube 150 and ball 120 are thus free to rotate. Because the interface surfaces in FIG. 8 are relatively smooth, there may be a tendency for some amount of slippage, particularly under heavy use so an alternate embodiment is illustrated in FIG. 9. The embodiment in FIG. 9 uses a series of bumps 205 on the spherical outer surface 204 of the ball 120. These bumps 205 mate with dimples 213 on the segmented spherical surfaces 212 of the split hub segments 202A-202D. In FIG. 9 the cinch ring 200 has been released enough and the segments 202A-202D expanded enough to clear the surfaces 212 so the control tube 150 and ball 120 can easily rotate relative to each other. On the other hand, FIG. 10 shows a “locked-in” position” wherein the bumps 205 are mated with the dimples 213. In the position of FIG. 10 the cinch ring 200 has clamped the hub segments 202A-202D against the ball 120. This provides a very positive grip and provides a wide range of adjustments.

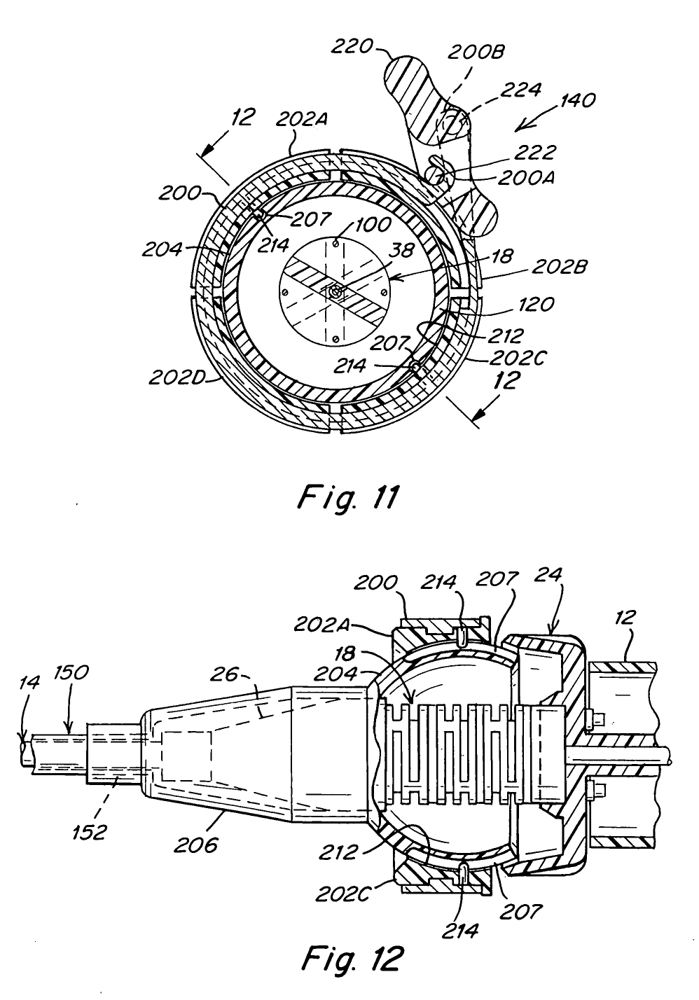

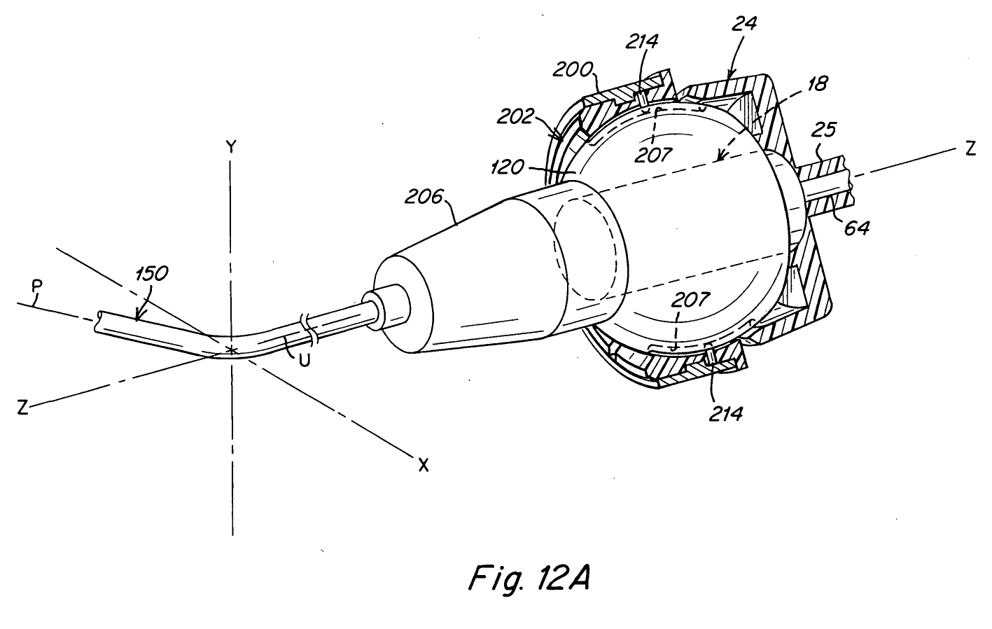

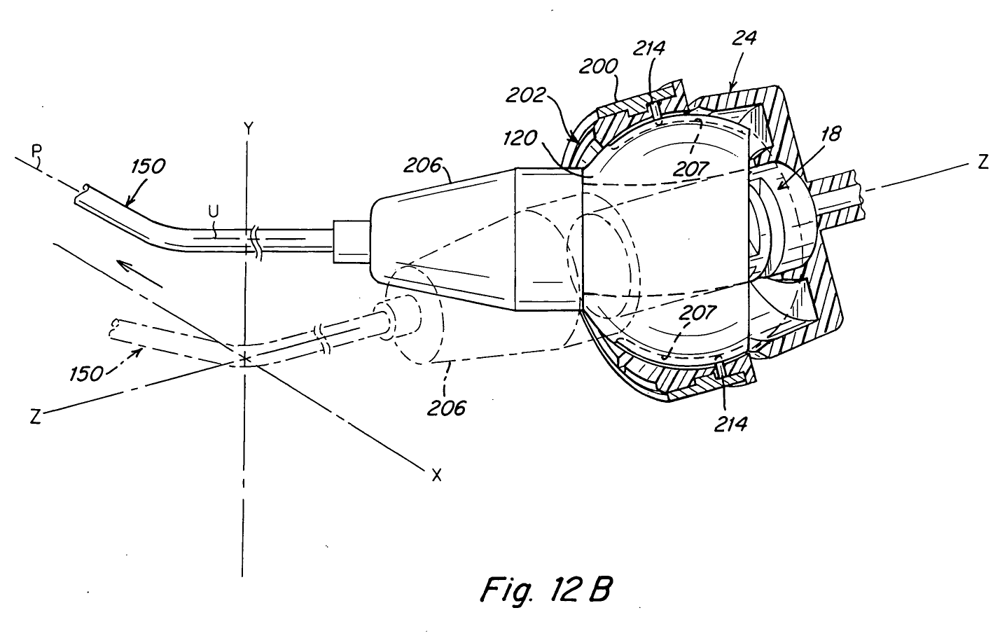

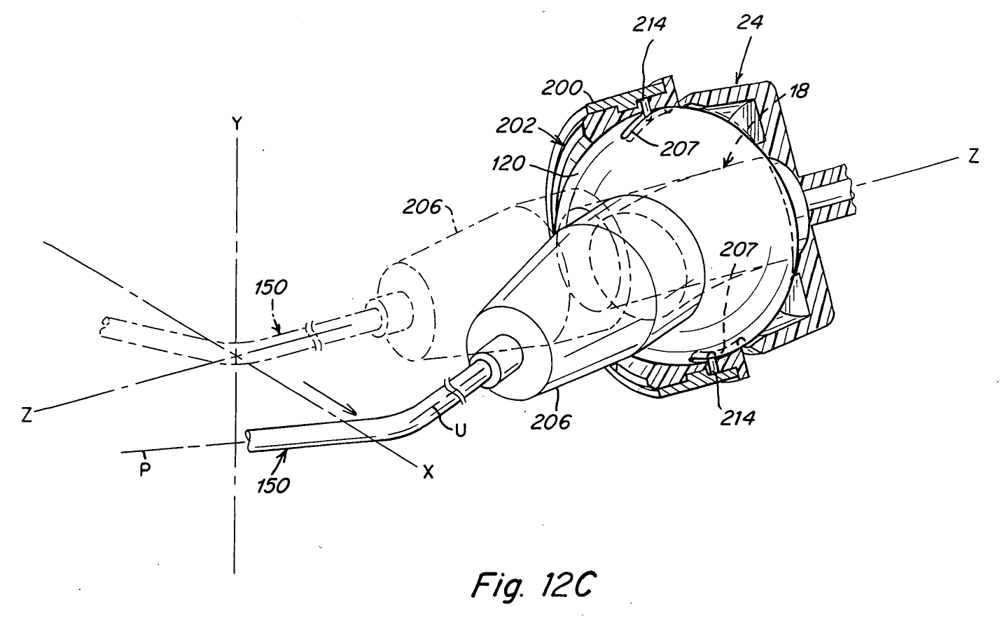

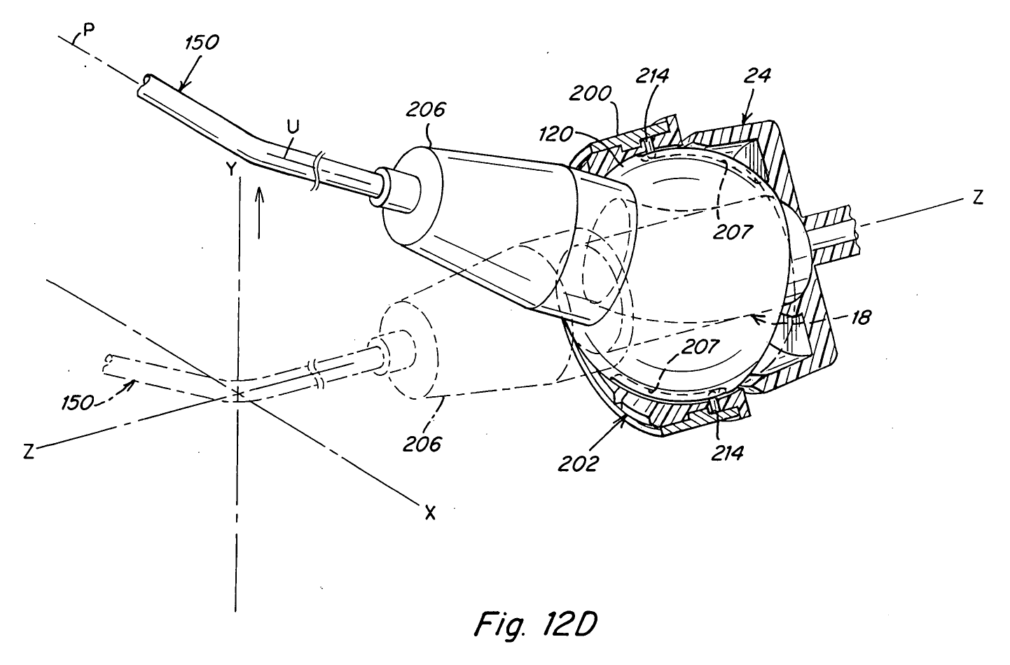

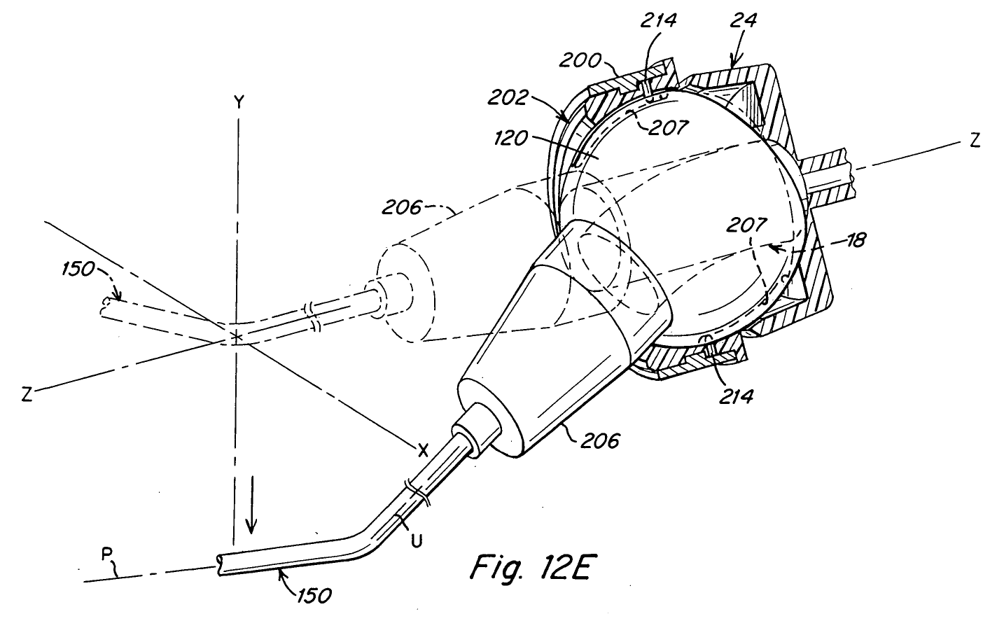

FIG. 11 is a cross-sectional view similar to that shown in FIG. 8 but showing an alternate embodiment of locking means along with the cinch ring 200 being in an alternate unlatched position. FIG. 12 is a cross-sectional view taken along line 12-12 of FIG. 11. FIGS. 12A-12E are a series of schematic fragmentary perspective views illustrating the oscillating motion of the ball member. FIG. 12A illustrates a neutral position with the control tube 150 disposed in the X-Z plane. FIG. 12B shows an oscillation in the X direction while FIG. 12C shows the opposite X direction motion. FIG. 12D shows an oscillation in the Y direction while FIG. 12E shows the opposite Y direction motion. In the alternate embodiment of FIGS. 11 and 12 the orientation of the control tube 150 is limited to two positions that are 180 degrees apart from each other in an X axis direction. The embodiment of the instrument shown in FIGS. 11 and 12 essentially holds the plane of the control tube 150 fixed, but enables an oscillation movement, via the ball member 120 in its socket, of the control tube 150 and, in turn, the distal part of the instrument. This motion, which is also referred to as an oscillation, is in the X and Y directions, as illustrated in FIGS. 12A-12E. By referring to an “oscillation” this means the interaction of the ball in the socket in essentially three dimensions, four positions of which are illustrated in FIGS. 12B-12E from the neutral position of FIG. 12A.

The combination of the opposed pins 214 in opposed slots 207 form a gimbal that, with respect to, for example, FIG. 12, allows motion (oscillation) of the ball and thus also the curved control tube 150, in both X and Y directions. However, this arrangement also prevents ball rotation such as in the direction R6 shown in FIG. 1. This thus allows the surgeon to fix one of the degrees of freedom of the instrument so that more concentration can be directed to other control actions of the instrument. This essentially fixes the plane of the curved tube.

The instrument can be set up for either left or right hand use by controlling the expansion of the cinch ring 200. In the embodiment shown in FIGS. 11 and 12 the plane of the control tube is essentially held in a fixed position, while at the same time allowing oscillation of the ball 120 in its socket (hub 202) in the X and Y directions. This does provide a controlled re-positioning of the distal part of the instrument. The lock release lever 220 is illustrated as having pins 224 having enlarged heads that allow cinch ring end 200B to be released from end 200A by a snap fit or other means. This allows the cinch ring 200 and at least hub segments 202A-202C to be expanded enough to allow restrictive pins 214 attached to hub segments 202A and 202C to be displaced from their respective slots 207 on the surface 204 of the ball 120. This, in turn, enables the ball 120 to rotate through 180 degrees until the pins 214 again engage the slots 207 on the opposite side of the ball 120. The pins 214 preferably have rounded heads and are diametrically opposed in hub 202. The pins 214 engage matching diametrically opposed slots 207 on the surface 204 of the ball 120.

After the ball 120 has been rotated through 180 degrees, then the ends of the cinch ring 200 can be reattached and the instrument is then ready for use. Theoretically the X and Y orientation of the diametrically opposed pins 214 and slots 207 can be at any convenient X, Y angle around the center of the ball and hub and act as gimbals that prevent rotation of the ball 120 in its socket and maintain a planar orientation of the bent tube while allowing the ball to oscillate within its socket in the hub. In the partially released position of the cinch ring 200 seen in FIG. 11 the cinch ring can be rotated by itself for left or right hand use to match the orientation of the curved control tube 150. In the partially released position the ball is loose enough to oscillate to allow adjustment of angle B1 to the desired bend angle B2 of the end effector. The cinch ring 200 can then be tightened and the angles locked in.

As mentioned previously, the neck of the ball, as well as the ball itself along with the curved control tube, is free to oscillate in both X and Y directions. It does not matter if the pins are on the X axis or at any number of degrees about the X and Y axes since they are diametrically opposed across the center of the ball and free to slide in the slots. The opposed pins are shown in FIG. 11 at a 45 degree position. This is primarily so as to not interfere with the gaps defined between the segments of the hub. On the other hand, the gaps in the hub and the support struts may be positioned so that the normal position of the curved control tube is directly in the X-Z plane (see FIG. 1). In that case the pins 214 would be opposed horizontally in FIG. 11. In that case even when the ball is rotated through 180 degrees the plane of the curved control tube is still in the X-Z plane, but directed in the opposite direction. The plane formed by the bent control tube has the same oscillation (bending) movements as if there were no slots and pins. The slots and pins just keep the ball from rotating.

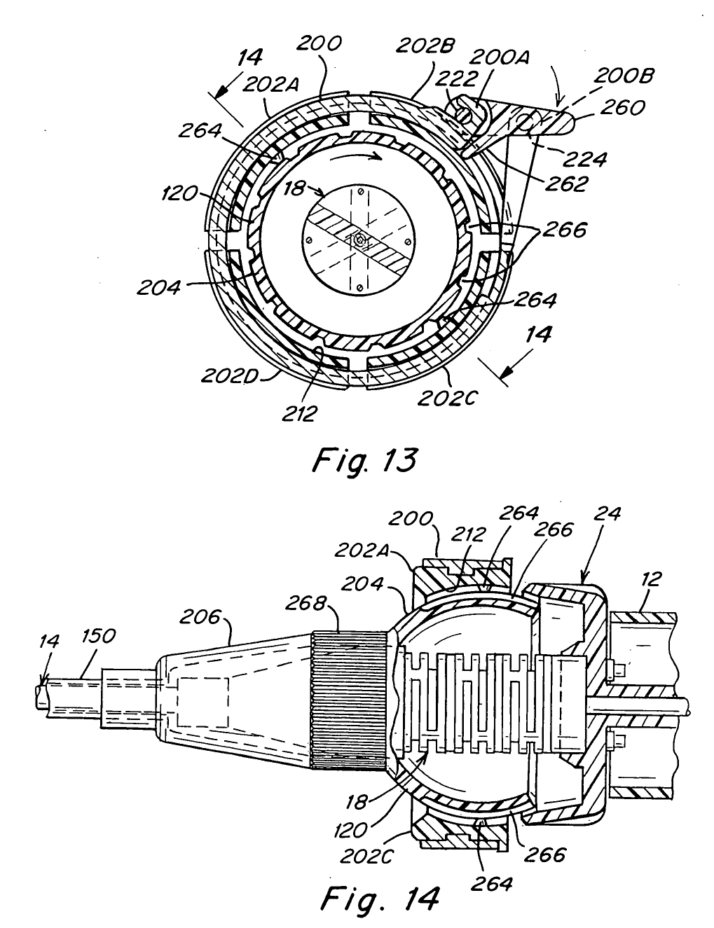

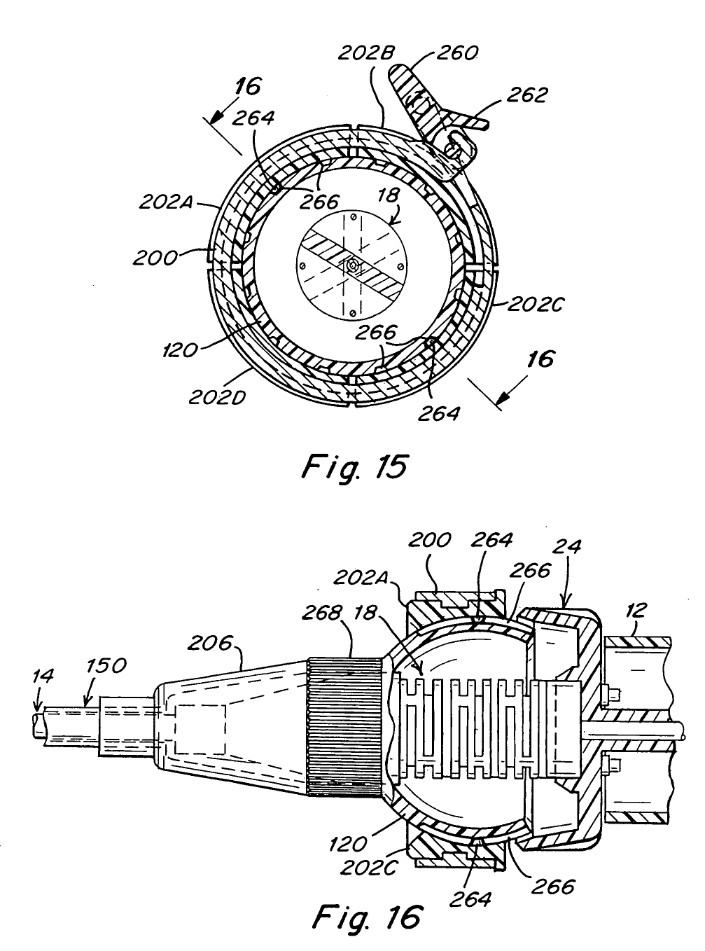

Reference is now made to another embodiment of the present invention illustrated in FIGS. 13-16. FIG. 13 is a cross-sectional view similar to that shown in FIG. 8 but showing the alternate embodiment of control tube and angle locking means with the lock released. FIG. 14 is a cross-sectional view as taken along line 14-14 of FIG. 13. FIG. 15 is a cross-sectional view similar to that shown in FIG. 13 but showing the angle locking mechanism in a locked position. FIG. 16 is a cross-sectional view as taken along line 16-16 of FIG. 15. FIG. 13 illustrates a control tube and locking mechanism wherein the rotation of the control tube 150 is limited to 30 degree preset intervals. Of course, other rotation intervals may also be used. Two diametrically opposed pins 264 in the shape of truncated cones are formed on the surface of split hub segments 202A and 202C and engage any two opposed grooves 266 of the twelve grooves 266 in the surface 204 of ball 120. This embodiment has some similarities to the embodiment shown in FIGS. 8-12 but includes more options as far as the rotation of the ball is concerned. In order to facilitate easier adjustment the sides of the grooves are tapered to match the taper on the pins and provide a cam surface that will raise the pins out of their grooves when the cinch ring is released and rotational force is applied to the knurled grip 268 on the neck portion 206 of the ball.

In this embodiment the lock/release lever 260 has been modified from that shown in FIG. 11 to allow more slack in the cinch ring 200 when the cinch ring is released. As is illustrated in FIG. 13, when the lever 260 is rotated to its' unlocked position the ends 200A and 200B of the cinch ring are spread apart enough to allow the pins 264 to cam out of the grooves 266 and they are thus able to slide across the surface 204 of the ball 120. A stop 262 on the lever 260 prevents the cinch ring 200 from opening enough to fall out of its' seat in the split hub 202. The lock/release lever 260 may also be used with the embodiment of FIGS. 8-12 to allow 180 degree adjustment of the ball without having to disconnect the ends 200A and 220 B of the cinch ring 200. FIGS. 13 and 14 show the control tube 150 and ball 120 being rotated, while FIGS. 15 and 16 show the tube and ball locked in place. As in the previous embodiment the pins 264 function as gimbals that allow the ball to oscillate but not rotate in the socket. See the previous description relating to FIGS. 8-12 for further details of the operation and control provided by this embodiment.

Reference is now made to the schematic illustrations shown in FIGS. 17A-17E. FIGS. 17A-17E shows diagrammatically the use of two instruments simultaneously, and as may be used in laparoscopic surgery. These diagrams illustrate the manner in which the tips of the respective instruments can be operated to raise or lower the distal part or end effector of each of the instruments to, in turn, provide enhanced control of the tip of the instrument. The controllable curved control tube arrangement is, in particular instrumental in allowing improved triangulation of the instruments so that there is a far less likelihood of collision between the respective instruments, both at the proximal and distal parts of the instruments. In each of FIGS. 17A-17E there are depicted the two instruments 10A and 10B inserted into the anatomy through the illustrated port 8. Also illustrated are the respective curved control tubes 150A, 150B and end effectors 16A, 16B. The instrument illustrated in FIGS. 17A-17E may be considered as the one shown in the first embodiment in FIGS. 1-10.

In FIG. 17A instrument 10A has a control tube 150A with an end effector 16A that may be considered as being oriented to the right and instrument 10B has a control tube 150B with an end effector 16B that may be considered as being oriented to the left. For the sake of discussions both of the curved control tubes may be considered as in the same plane, and more particularly in the X-Z plane. FIG. 17A also shows by arrows S the possible linear motion of the instrument shafts. Pivoting may also be controlled by the surgeon at the port 8. Both shafts pass through a single port 8 of entry or cannula and cross over each other, as illustrated. This arrangement places the end effectors 16A and 16B at their tips in close proximity for triangulation of the instruments, while spacing the instrument handles far enough apart to avoid interference or collisions between the instruments. Again, the instruments are free to slide in and out of the port of entry a certain amount under user control (arrows S).

Reference is now made to additional controls of the instruments 10A and 10B as depicted in FIGS. 17B-17E. In FIG. 17B the handle of the instrument 10A has been orbited clockwise about axis Z in the direction of rotational arrow R4. This results in a left side tip down of the orientation of instrument 10A. This tipping down of the end effector is illustrated by rotational arrow R5 in FIG. 17B. In FIG. 17B the instrument 10B is in the same position as in FIG. 17A. This rotation in direction R5 can also be accomplished by rotating the ball 120 relative to the handle. FIG. 17C shows how orbiting the instrument 10A counterclockwise about axis Z results in a left side tip up of the orientation of the end effector 16A. FIG. 17C shows the handle rotation by arrow R4 which is in the opposite direction to that shown in FIG. 17B, and the resulting upward rotation of the tip of the instrument 10A as illustrated by rotational arrow R5. This rotation in direction R5 can also be accomplished by rotating the ball 120 relative to the handle. In FIG. 17C the instrument 10B is in the same position as in FIG. 17A. Other illustrations are possible in which instrument 10B can be rotated, or both instruments concurrently rotated to re-position the tip of the instruments. FIG. 17D shows how pivoting instrument 10A upward, in the direction of arrow M1 along the Y axis results in a left side tip down orientation. FIG. 17E shows how pivoting instrument 10A downward, in the direction of arrow M2 along axis Y results in a left side tip up orientation. Both of these motions move the tip of the instrument 10A out of the X-Z plane. Other illustrations are possible in which instrument 10B can be also tipped up or down, or both instruments concurrently re-positioned at the tip of the instruments. Moreover, either the handle or ball of the respective instruments may be rotated to control other instrument re-positioning.

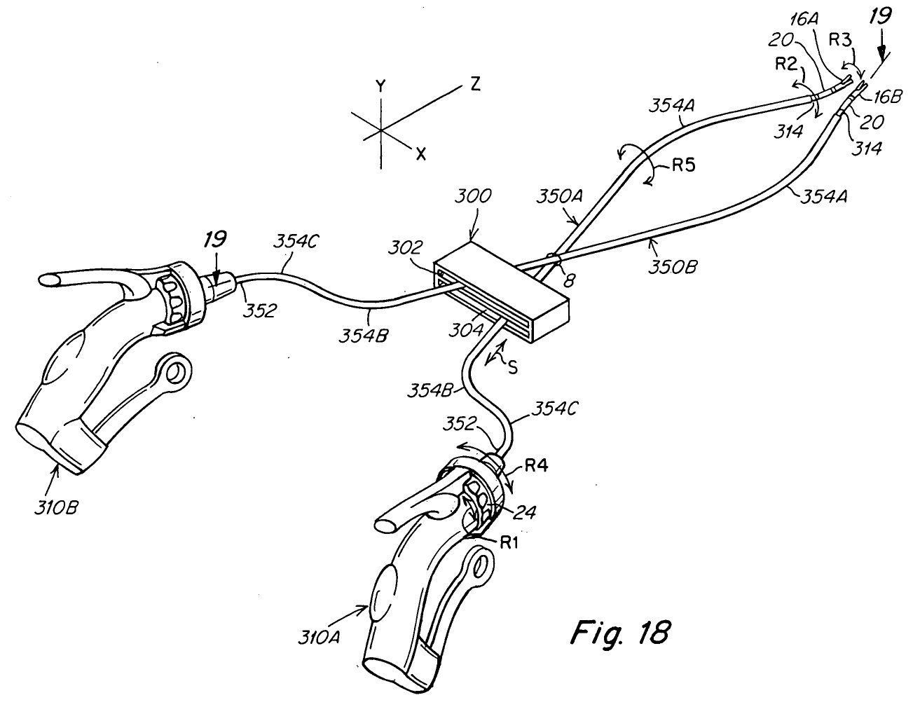

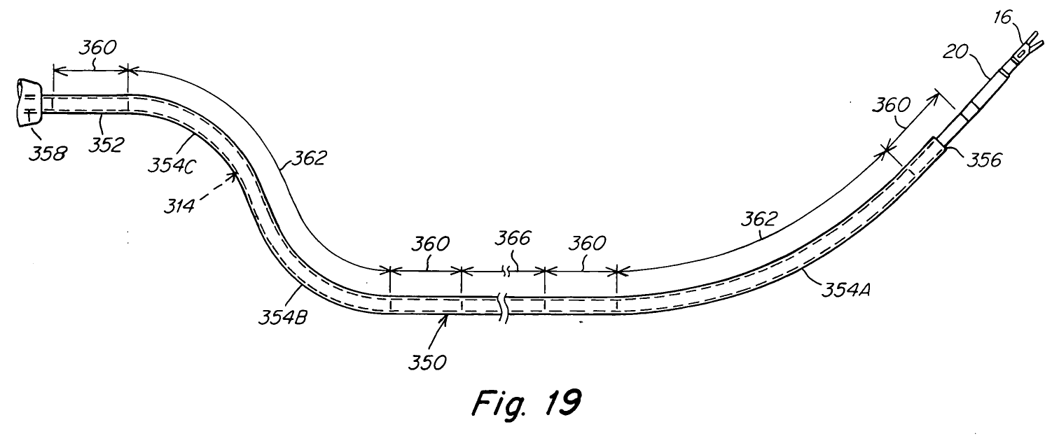

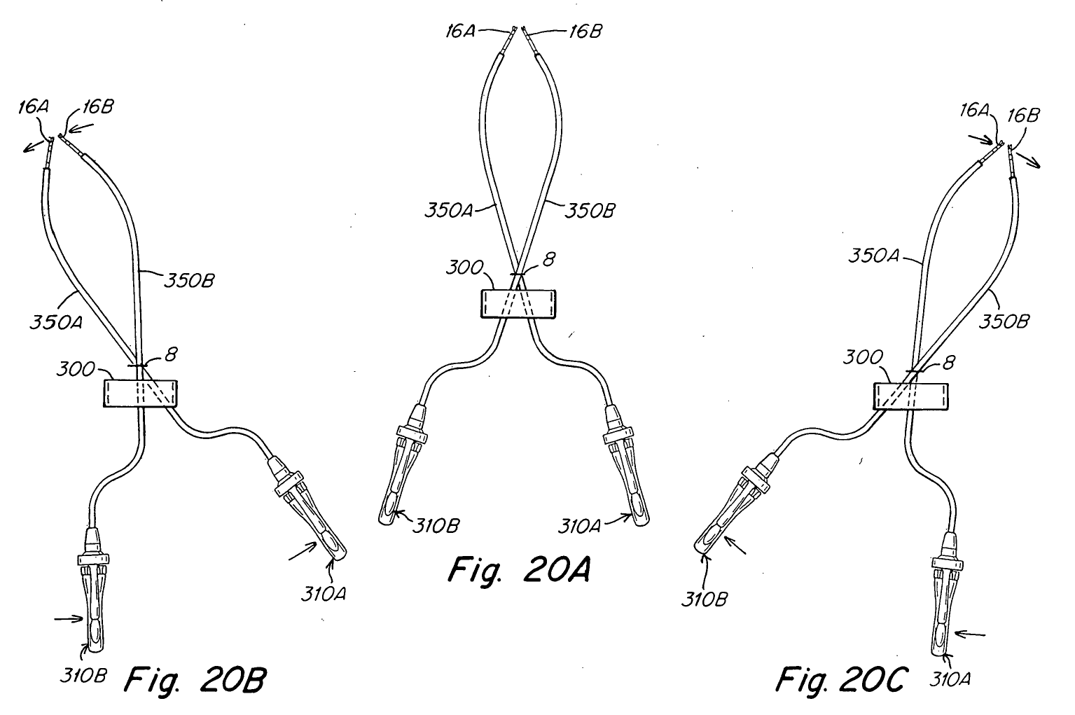

FIG. 18 is a perspective view of an alternate embodiment of instrument in use. FIG. 19 is a fragmentary cross-sectional view of the instrument shaft and control tube taken along line 19-19 of FIG. 18. FIGS. 20A-20C, 21A-21C and 22A-22C are diagrammatic respective plan, rear and side views showing different ways of manipulating the instruments shown in FIG. 18 during a surgical procedure.

FIG. 18 illustrates a pair of instruments 310A and 310B each having three curves in their respective control tube 350A, 350B. These particular instruments are shown as being supported through the guide block 300. For supporting the instruments 310A and 310B the guide block 300 has separate parallel upper and lower through slots 302, 304. The guide block 300 is meant for fixed positioning adjacent to but just outside of the incision port 8. The instrument 310A is supported through the lower slot 304, while the instrument 310B is supported through the upper slot 302. The instruments may also be supported through the alternate slots. In this embodiment two of the three bends in each instrument are disposed proximal to the guide block, while a single bend is disposed distal to the guide block. Each of the instruments illustrated in FIG. 18 may be considered as substantially the same as the one shown in the first embodiment in FIGS. 1-10. FIG. 18 also shows the end effectors 16A and 16B associated respectively with the instruments 310A and 310B.

Considering by way of example instrument 310A, the various arrows show the different motions that can be controlled. Arrow R1 depicts the rotation at the rotation knob 24. This causes the inner instrument shaft to rotate as illustrated by the arrow R2 at the distal end of the shaft axis and distal bendable member 20, and, in turn, rotation R3 at the very distal tip of the end effector 16A. Arrow R4 at the handle end of the instrument depicts a rotation of the handle by the user of the instrument. This translates into a rotation of the curved control tube 350 as depicted by arrow R5. Double-headed arrows S illustrate the possible motion by the surgeon of either instrument in an inward-outward direction relative to the incision port.

The most distal curve 354A serves to help triangulate the instrument tips as in the previous embodiment and the two more proximal curves 354B and 354C allow for up/down translation of the instrument tips without pivoting up and down at the incision port. This up/down movement is possible by either rotating the respective handles or the respective balls of each instrument. The guide block 300 holds the instrument shafts in two parallel planes greatly reducing the likelihood of a collision between the instrument shafts or control tubes. The guide block 300 is situated just proximal of the cannula port 8, and the respective instrument shafts 314 and their associated control tubes 350 pass through and are slidable (arrow S) in these slots 302, 304. Instrument 310A may be considered as having a right oriented curve 354A and instrument 310B having a left oriented curve 354A. Once again, the directional arrows R1-R5 indicate similar motions as the embodiment of FIGS. 1-8.

FIG. 19 is a fragmentary plan view of the instrument shaft and control tube as seen along line 19-19 of FIG. 18. This construction may be substantially the same as previously shown and discussed in connection with FIG. 2 herein. In order for the instrument shaft 314 to be able to rotate within the rigid bent portions 354A-354C of the control tube 350, flexible sections 362 have been added to the instrument shaft 314, similar to that illustrated in FIGS. 2 and 3. A short rigid section 358 of the instrument shaft 314 is attached to the adapter and is free to rotate within the proximal section 352 of the control tube 350. The rigid section 358 may have a shaft filler with a lumen for receiving the inner shaft tube, as well as grooves for accommodating the bend control cables. The rigid section 358 is attached to the flexible section 362 by a connector 360 that is preferably a short piece of stainless steel tubing about 2 inches long that is force fit or otherwise bonded to the flexible plastic tubing 362. At the distal end 356, the flexible section 362 is connected by another cylindrical connector 360 to the distal bendable member 20 which is articulated by the bend control cables. The instrument shaft may also include a rigid center section 366 that extends along the straight length of the control tube that passes through the guide block 300. The distal end of the flexible section 362 is then connected to the distal bendable member. If the rigid tube is first formed in its bent condition, then virtually all sections of the instrument shaft are to be flexible so that the instrument shaft can pass through the control tube 350. On the other hand, the instrument shaft can be inserted in an initially straight control tube with the control tube being later bent into the shape as shown in FIG. 18.

FIGS. 20A-20C; 21A-21C and 22A-22C are respective diagrammatic plan, rear and side views showing different ways of manipulating the instruments shown in FIG. 18 during a surgical procedure. FIGS. 20A-20C diagrammatically shows by a plan view how lateral translations of the instrument tips occur by pivoting the instruments 310A and 310B at the incision (port 8). FIG. 20A shows the instruments 310A and 310B at a neutral position and basically symmetric relative to the guide block 300. FIG. 20B shows both instrument handles pivoted to the right causing a corresponding movement of the end effectors to the left. FIG. 20C shows both instrument handles pivoted to the left causing a corresponding movement of the end effectors to the right. This pivoting occurs by moving the straight section of the control tube passing through the guide block 300 of each instrument laterally in the respective slots in the guide block.

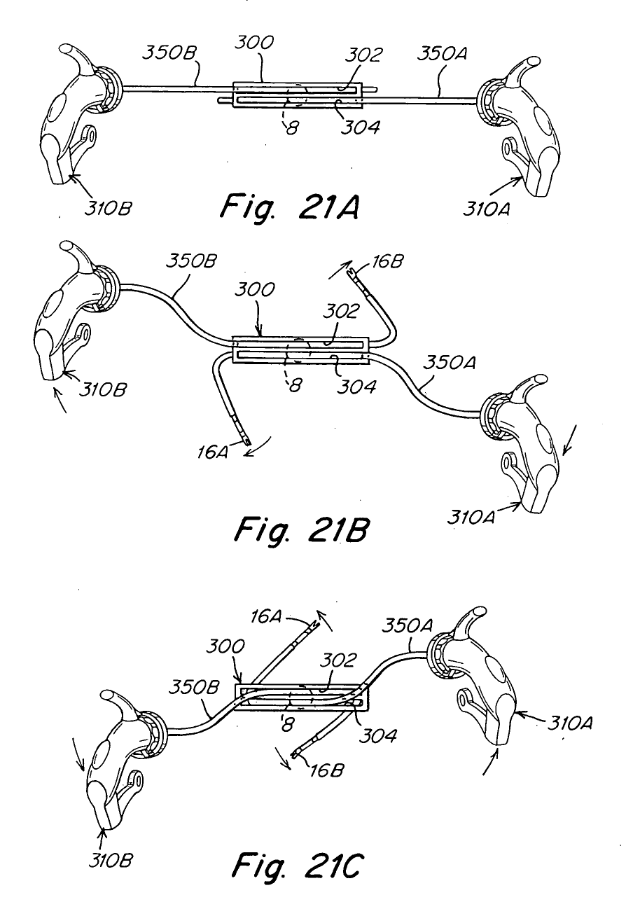

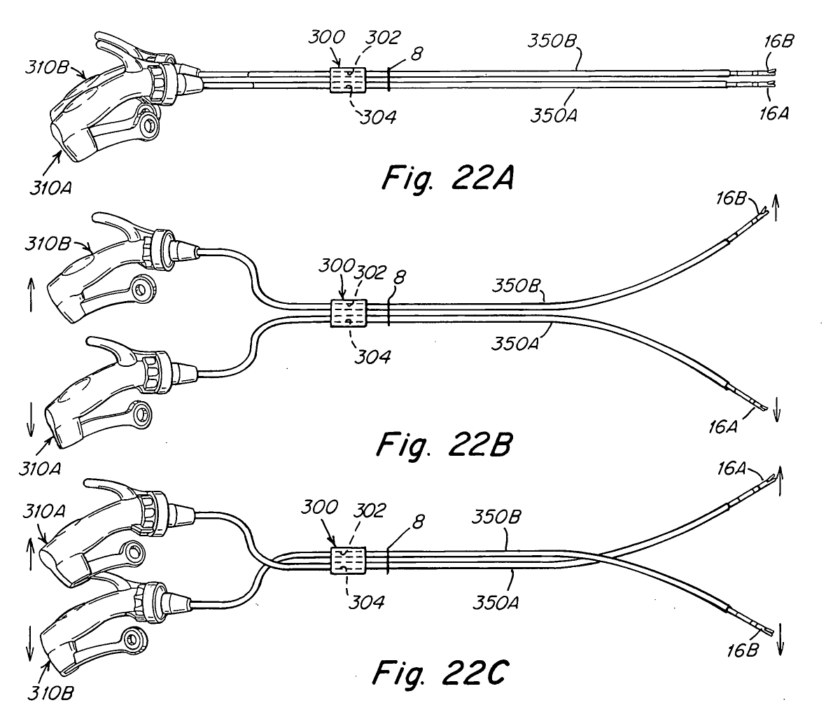

FIG. 21A-21C diagrammatically show how an up/down translation of the instrument is performed. An advantage of this arrangement is that an upward movement of the handle causes upward translation of the tip of the instrument and a downward movement causes downward translation of the tip of the instrument. This is due to the fact that there are preferably two proximal bends and an odd number of total bends in the control tube 350, illustrated in FIG. 19. In FIG. 21A the instruments may be considered as disposed in a like plane with the handles of the respective instruments initially symmetric and at the same position heightwise. In FIG. 21B the instrument 310A is moved downwardly causing the end effector 16A to likewise move downwardly. In FIG. 21B the instrument 310B is moved upwardly in a pivoting manner causing the end effector 16B to likewise move upwardly. An opposite action is depicted in FIG. 21C, wherein the instrument 310A is moved upwardly causing the end effector 16A to likewise move upwardly, and the instrument 310B is moved downwardly causing the end effector 16B to likewise move downwardly. This pivoting action is caused by, for example, as the handle of instrument 310A moves downwardly, a downward rotation occurs at the bend 354C which, in turn, causes an upward movement at the bend 354B. This action via the rigid control tube 350 causes the third bend 354A to rotate the tip of the instrument downwardly. Thus a downward motion at the handle causes a corresponding downward motion at the tip of the instrument. This is important in providing the surgeon with a feel that the directional movement at the handle translates into a same direction movement at the end effector. FIGS. 22A-22C in effect corresponds respectively to the positions shown in FIGS. 21A-21C. FIGS. 22A-22C are diagrammatic side views of the up/down translation of movements depicted in respective FIG. 21A-21C.

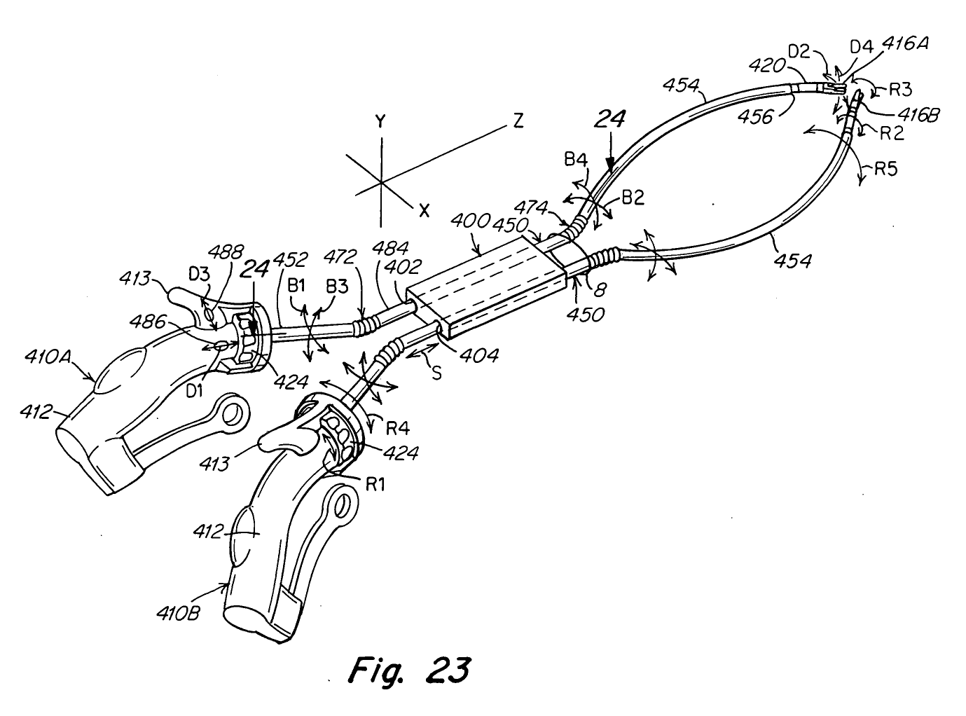

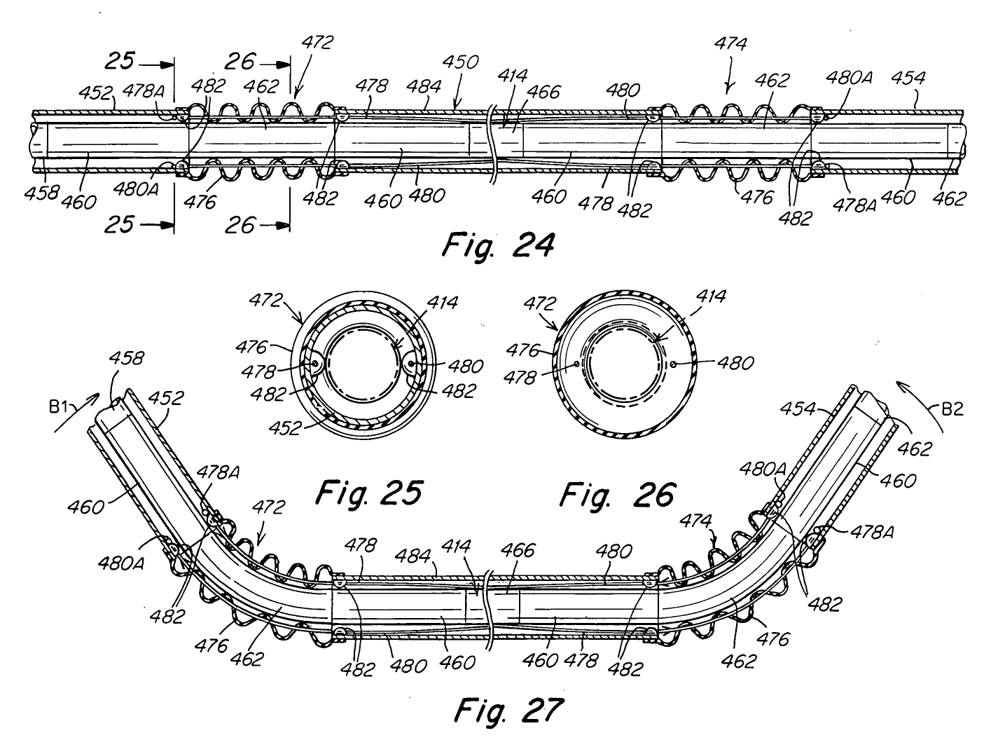

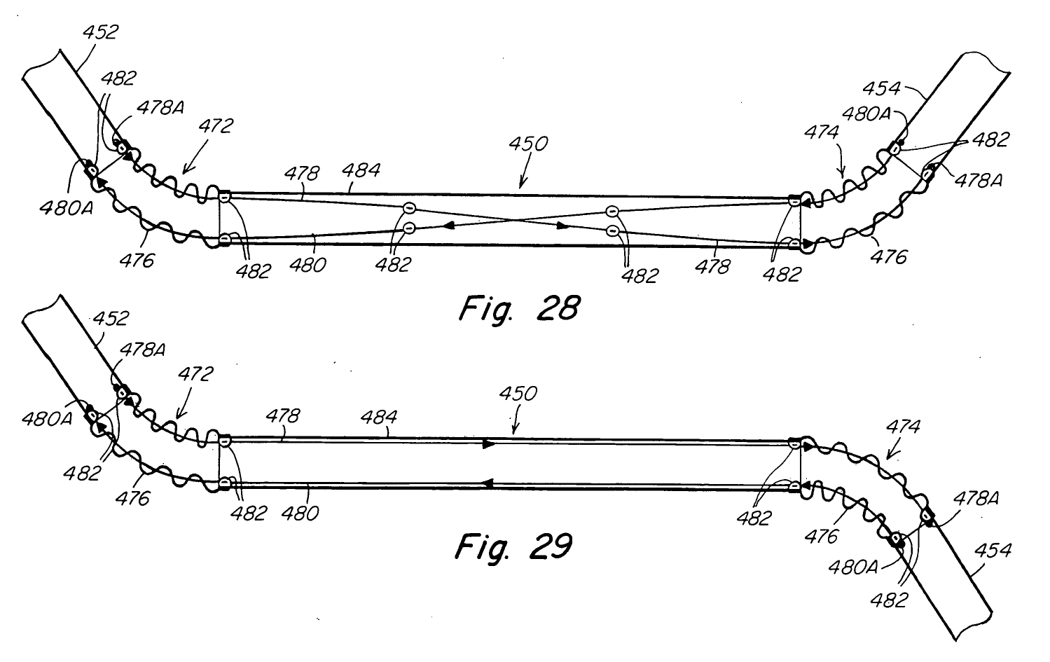

Reference is now made to another embodiment of the present invention as illustrated in FIGS. 23-29. FIG. 23 is a perspective view of this alternate embodiment of the instrument in use. FIG. 24 is a cross-sectional view of the articulation sections and mid portion of the control tube as taken along line 24-24 of FIG. 23. FIGS. 25 and 26 are cross-sectional views as taken respectively along lines 25-25 and 26-26 of FIG. 24. FIG. 27 is a cross-sectional view similar to that shown in FIG. 24 but showing the articulation sections in a bent condition. FIG. 28 is a schematic view of FIG. 27. FIG. 29 is a schematic view similar to that shown in FIG. 27 but with an alternate arrangement of the cabling means.

In the embodiment shown in FIGS. 23-29 the curved control tube 450 of each instrument is constrained by an over tube 400 which limits the motion of the control tube to a sliding motion in the direction of arrows S and/or an axial rotation indicated by arrows R4 and R5 in FIG. 23. The advantage of this embodiment, in comparison to earlier embodiments, is that the left handle is operating the tool on the left and the right handle is operating the tool on the right. The guide shafts 450 pass through respective guideways 402 and 404 of the over tube 400, disposing the respective rigid sections 484 of the respective tubes in parallel. It is noted in this embodiment that the instruments do not cross each other as in previous embodiments. This instrument system is also characterized by the instruments avoiding collisions due to their placement and construction. Each of the control tubes 450 includes articulation sections 472 and 474 at each end of the rigid section 484 of control tube 450. These articulation sections (bendable members) provide additional degrees of freedom while keeping the instrument tips and the instruments themselves separated from each other to avoid collisions. The articulation sections 472, 474 are connected to each other by cables or alternately a cable drive system, as will be described in more detail hereinafter.

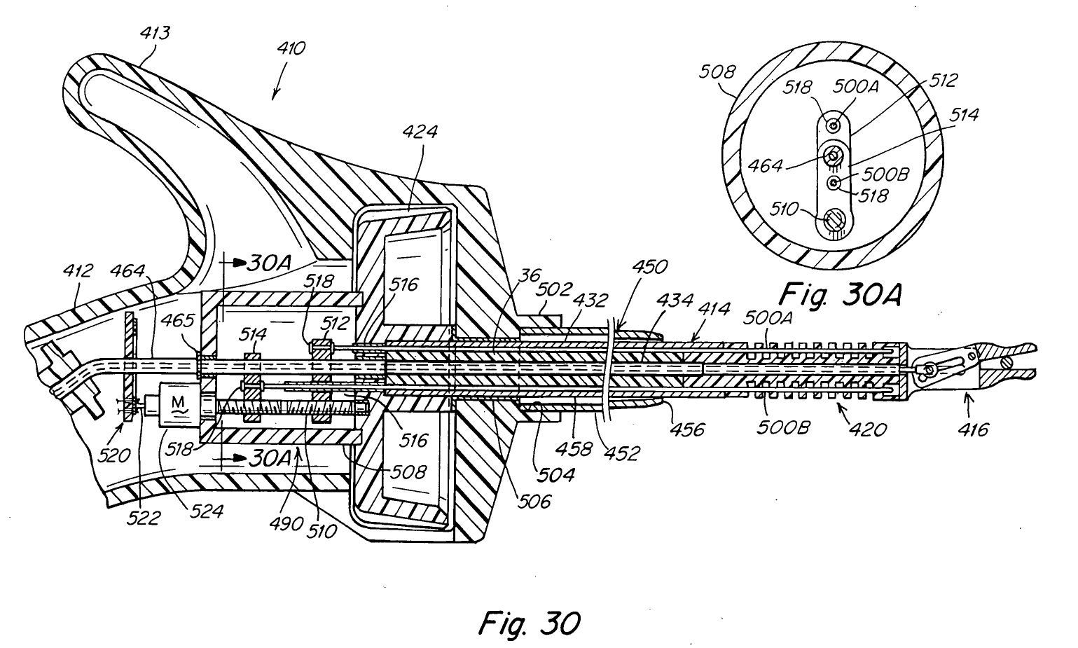

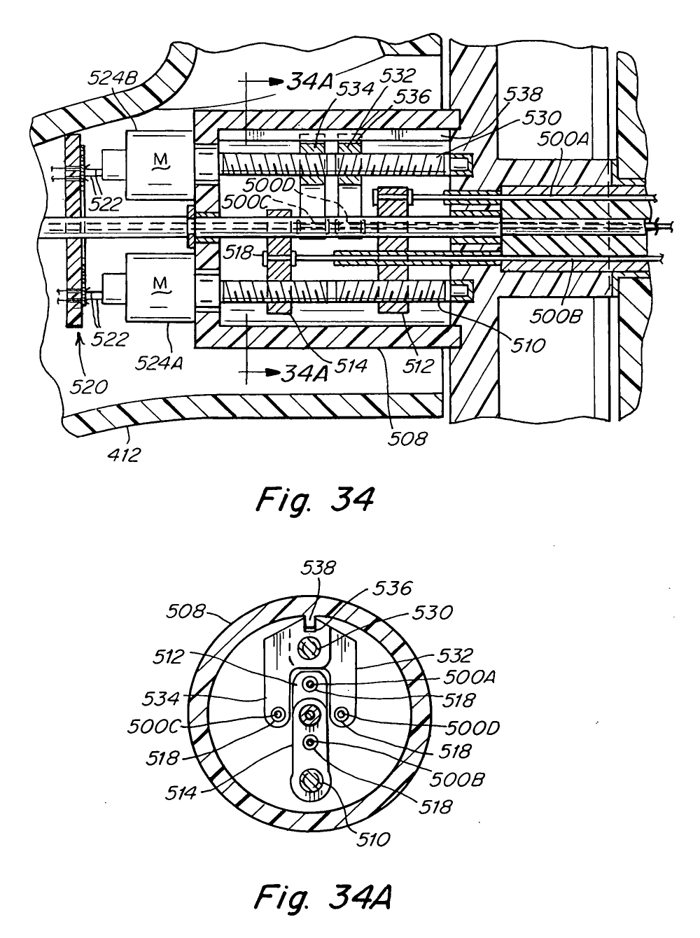

Rigid bend portions 454 extend respectively from the articulation sections 474 to the instrument tips and are used to provide triangulation of the end effectors 416A and 416B. In this embodiment the horn 413 has been shortened in comparison to, for example, the embodiment shown in FIG. 1, and the proximal bending member has been replaced by a push/pull cable drive mechanism 490 (see FIGS. 30-34) that is operated by rocker switches 486, 488 as best illustrated in FIG. 23 on the handle housing of instrument 410A. Like switches may also be provided on the handle housing of instrument 410B. The activation of the switch 486 in the direction of double arrow D1 controls the side-to-side movement of the end effector 416A, as illustrated by the double arrow D2 in FIG. 23. The activation of switch 488 in the direction of double arrow D3 controls the up/down movement of the end effector 416A, in the direction of double arrow D4. The bending actions of the distal bending member 420 are controlled with cables 500A-500D as will be discussed in more detail hereinafter. Although four degrees of freedom are illustrated herein, it is contemplated that alternately only two degrees of freedom might suffice because the surgeon can also rotate the end effector either by rotating the handle of the instrument or by using the rotation knob on the instrument. The four degrees of freedom are possible when using an arrangement such as that illustrated in FIG. 34 wherein two control motors are used, as will be described in more detail hereinafter.

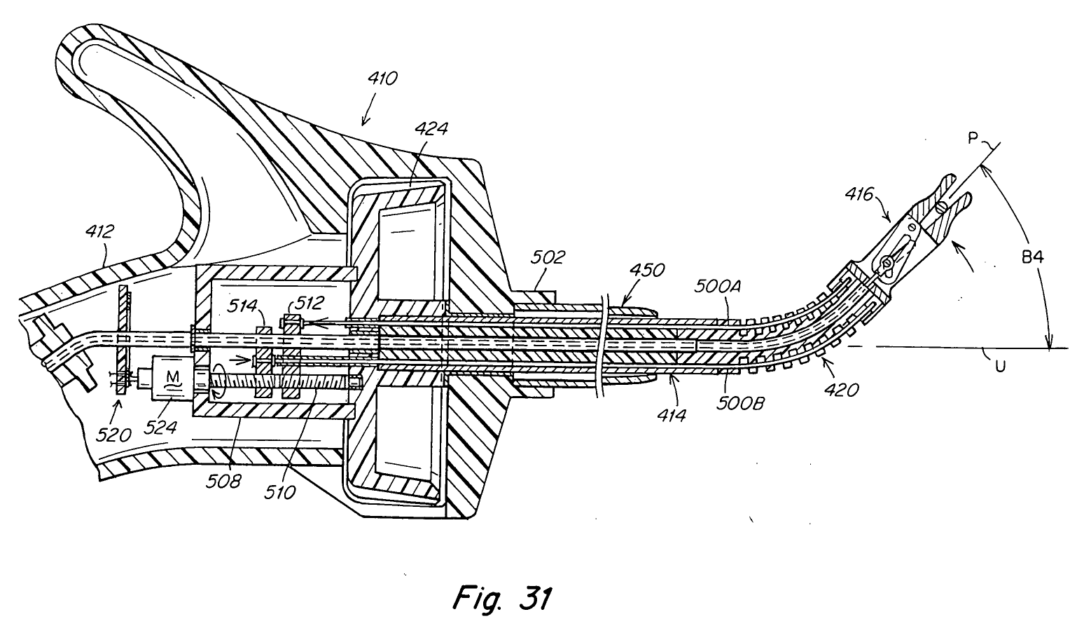

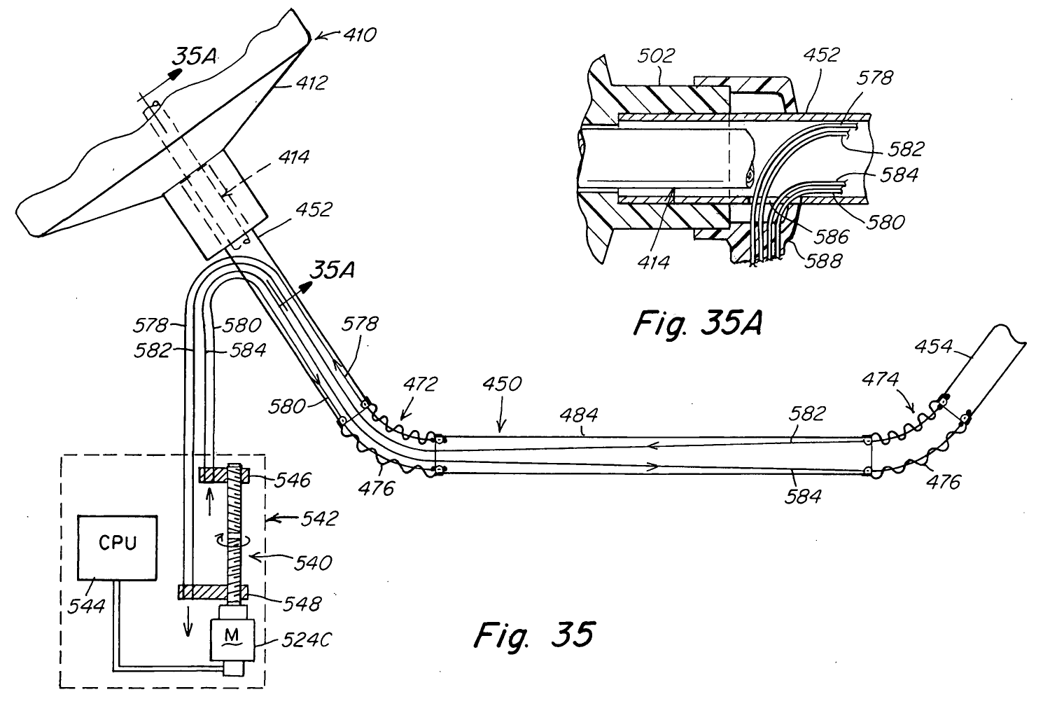

There are a number of different controls that can be exercised with the instrument system illustrated. For example, one can use the switch 486 to move the end effector 416A to the left in the direction of arrow D2 and use the rotation knob 424 to raise (orbit) the end effector in the direction of arrow D4. This movement controlled by the rotation knob 424 in the direction of arrow D4 is enabled when the distal bendable member is in a bent condition such as shown in FIGS. 31-33. A CPU controller can be used, such as shown in FIG. 35, to translate signals from switches 486, 488 to control the motors. Also, one can use a cable drive mechanism with just two cables to bend the distal bendable member and a corresponding rotational drive mechanism independent from rotation knob 424 to position the end effector in both X and Y axes.

Another alternative embodiment can use an electronic control for the cabling. This is particularly advantageous when the two motor arrangement is used to control four cables. As indicated before the control with the embodiment using the motors for the proximal section provides an orbiting effect when the distal bendable member is bent. However, it is desirable in the two motor arrangement to be able to control the tip of the instrument to rotate about the axis P rather than orbit about axis S. Since the unitary slotted proximal bending member has been replaced by the motor and cable drive arrangement described herein a CPU or the like is used to control the cabling 500A-500D as the rotation knob is turned in order to keep the end effector rotating about axis P and not axis S.

FIG. 24 is a fragmentary, somewhat schematic cross-sectional view of the control tube and shaft taken along line 24-24 of FIG. 23. For the sake of simplicity only two cables 478 and 480 are shown. However, four cables are preferred disposed at 90 degree intervals in order to provide a full 360 degree control between the articulation sections 472, 474. The control tube 450 has a short rigid section of tube 452 affixed to the hub 502 at the distal end of the handle 412, as illustrated in FIG. 30. The tube 452 is connected at its distal end to a first articulation section 472 which consists primarily of a bellows 476 with cables 478 and 480 passing through diametrically opposed clearance holes in the bellows. A sheath (not shown) may be used along the length of the articulation section (about the bellows) to seal off bodily fluids and to prevent them from entering the cable openings. The cables 478, 480 are secured respectively at anchors 478A and 480A at the proximal end of the articulation section 472 and at the distal end of the articulation section 474. The cables 478, 480 are supported by a series of cable guides 482. The cables extend via guides 482 at the distal end of tube section 452 and pass through guides 482 along their paths as they rotate 180 degrees around the inside of the middle rigid section 484. The cables then pass through the second articulation section 474 and another set of guides 482 affixed to the proximal end of curved section 454 and are then attached at anchors 478A and 480A. The anchors may be formed in a number of different ways.

The instrument shaft 414 which passes through the control tube may be constructed, starting from the proximal end, of a short rigid section 458 that is seated in the rotation knob 424, as depicted in FIG. 30. The section 458 is then joined by connector tube 460 to a first flexible section 462 that is coextensive with the bellows 476 of the first articulation section 472. Sections of the tubing 460 further interconnect the rigid portion 466 and the more distal flexible section 462 that is coextensive with the bellows 476 of the second articulation section 474. The latter tube 462 connects with the rigid curved section 454 and end effector 416 by connector tubes 460 as in the previous embodiments that have been described herein.

FIG. 27 is a cross-sectional view similar to FIG. 24 that shows the control tube articulation sections 472, 474 being bent in the direction of arrows B1, B2. FIG. 28 schematically depicts the manner in which the cables 478, 480 reverse the bend. FIG. 29 shows an alternate arrangement in which the cabling is in parallel alignment and the bend at 474 is in the same direction as the bend at 472. In order to get a full 360 degree control between the articulation sections 472, 474 another set of cables and guides (not shown) are used to control the orthogonal B3, B4 movements, as shown in FIG. 23.

FIG. 30 is a somewhat schematic fragmentary cross-sectional view of one embodiment of a cable drive mechanism 490 in a neutral position. For the sake of simplicity a two degree freedom of movement is shown with only one motor shown and without depicting any curved tube. The embodiment shown in FIG. 30 can be used for the system of FIG. 23, or can be used for a single instrument that is to be controlled. In the embodiment shown in FIG. 30 the instrument shaft 414 is illustrated supported at the proximal end at the rotation knob 424, extending through the control tube 450 and having the distal end couple via the distal bendable member 420 to the end effector 416. Only a short section of the control tube 450 is illustrated in FIG. 30. The motor 524 is mounted on a housing 508 which is attached to the proximal side of the rotation knob 424 in place of the previously used hub 25. The housing 508 and knob 424 are rotatably mounted on center wire conduit 464 and restrained longitudinally by e-ring 465 and bearing sleeve 506 in the hub 502

The motor 524 is electrically connected by a rotary connector 520 and brushes to a CPU (not shown) and switch 486 and/or switch 488. The motor drive includes a double screw thread on shaft 510 and two followers 512 and 514 which are driven in opposite directions to each other when the motor is activated. Thus, the respective threads on the shaft, for example, may be left and right hand threads. The followers are guided by clearance holes through which center wire conduit 464 passes in order to keep them from rotating when being driven by threaded shaft 510. The cables 500A and 500B are anchored to the followers at 518 and supported by PEEK tubes 516 before entering the first section of shaft filler 36 in instrument shaft section 458. The short rigid shaft section 458 is made up of outer shaft tube 432, inner shaft tube 434 and shaft filler 36 that is disposed between the tubes 432, 434. The control tube 450 is permanently connected in seat 504 of the hub 502 and is not adjustable since there is no proximal bending member or ball and neck. Along most of its length the control tube has a sufficient clearance for the connector tubes along different sections of the instrument shaft 414 but at its distal end 456 it may taper inward to keep out bodily fluids and provide a bearing surface to steady the end effector in use.

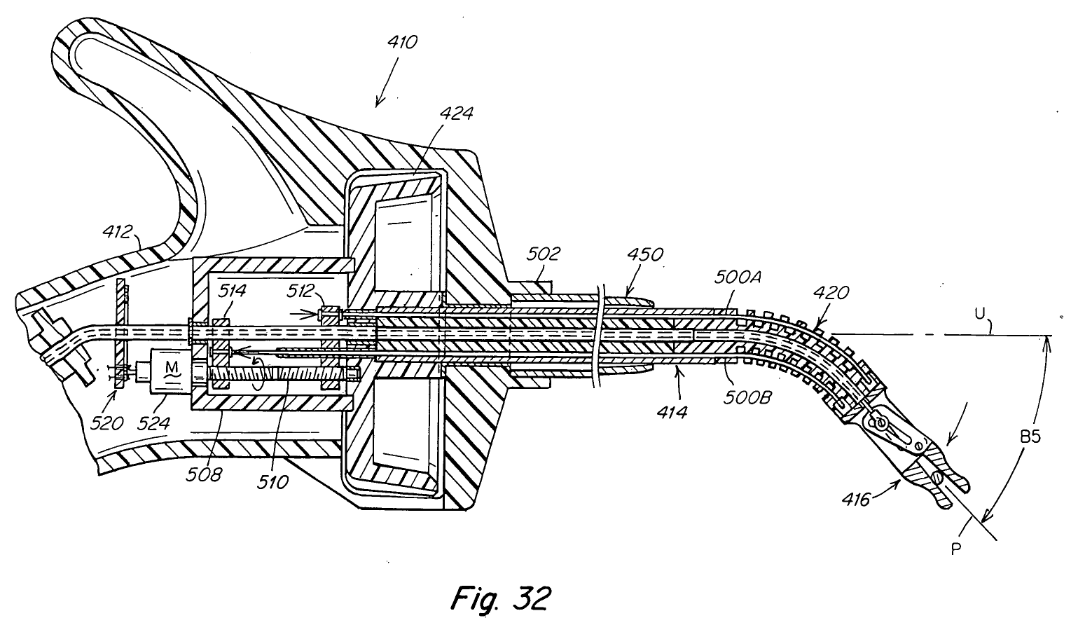

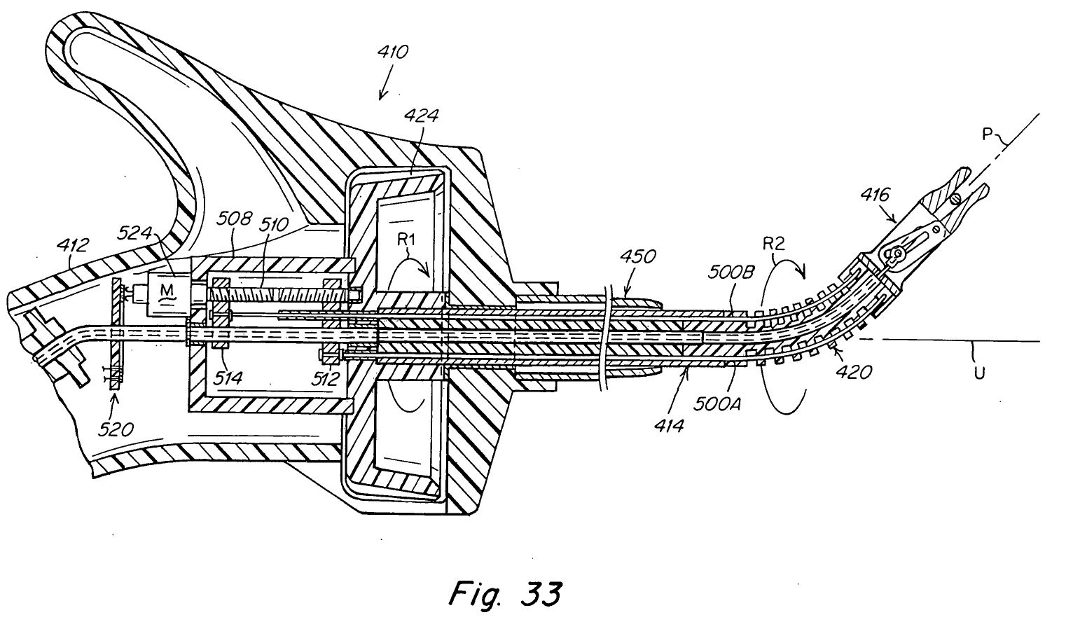

FIG. 31 is a schematic view similar to that shown in FIG. 30 but illustrating the drive mechanism bringing the followers 512, 514 toward each other and translating the cable motion into an upward bend B4 at the distal bendable member 420. FIG. 32 shows the followers 512, 514 moved apart from each other and the resulting downward bend B5 at the distal bendable member 420. FIG. 33 shows how rotating the rotational knob 424 in the direction of arrow R1 results in a rotation or orbiting at the distal bendable member in the direction of arrow R2. In other words, and with reference to FIG. 33, when the rotation knob is rotated the housing 508 rotates therewith while the motor 524 is maintained in contact with the rotational connector 520. This rotation of the knob 424 does not rotate the end effector about axis P, but instead orbits the end effector as indicated by arrow R2 in FIG. 33, and which is in a rotational direction in and out of the paper in FIG. 33 while the distal bendable member is in a bent condition. As stated before, this orbiting action can be overcome by the use of a CPU or other electronic control of the cabling that would be independent of the rotation knob rotation.

In FIGS. 30-33 with the use of only a single control motor the tip control is not in three dimensions, but instead only in two dimensions. However, FIG. 34 now illustrates a cable drive mechanism with four degrees of freedom. To accomplish this an additional motor drive and additional followers have been added along with two more cables. Motor 524A drives threaded shaft 510, followers 512 and 514, and cables 500A and 500B. Motor 524B, on the other hand, drives threaded shaft 530, followers 532 and 534 and cables 500C and 500D. As can be seen in FIG. 34A, the followers 532 and 534 are disposed off center and are guided by slots 536 along a rib 538 on the housing 508 to keep them from rotating along with the threaded shaft 530. This is one way to compensate for the automatic cable adjustments previously made by the proximal bending member. As the rotation knob is turned that keeps the end effector rotating about its' axis P. A CPU mutually driving motors 524A and 524B can mimic the same operation.

FIG. 35 is a schematic view of a motor driven cabling system 540 for the articulation of sections 472 and 474. FIG. 35A is a cross-sectional view as taken along line 35A-35A of FIG. 35. For the sake of simplicity only a two degree of freedom system is illustrated. However, it is understood that a four cable system can also be used. Another motor drive and set of cables would be used to achieve a four degree of freedom system. Due to limited space available in the handle it is contemplated that a peripheral unit 542 would be connected by electrical cables to the distal end of the instrument handle 412. A motor 524C is connected to a CPU 544. The followers 546 and 548 are connected to two cables each in a push pull relationship. As shown in FIG. 35, cables 580 and 584 are connected to follower 546 and cables 578 and 582 are connected to follower 548. This push/pulls arrangement enables the appropriate portions of the articulation sections to affect a reverse bend as shown. Alternately, the cabling can be set to enable a bend in the same direction. The cables 578,580, 582 and 584 can be exited from the guide shaft at port 586 and then enclosed in a strain relief housing 588 along with the electrical wires from the strain gauges and connected to the peripheral unit 542 without impeding the movement of the handles.

Having now described a limited number of embodiments of the present invention it should now be apparent to one skilled in the art that numerous other embodiments and modifications thereof are contemplated as falling within the scope of the present invention as defined by the appended claims.

What is claimed is:

1. A medical instrument comprising: an instrument shaft having proximal and distal ends; a tool for performing a medical procedure; a control handle; a distal motion member for coupling the distal end of the instrument shaft to the tool; a proximal motion member for coupling the proximal end of the instrument shaft to the control handle; actuation means extending between the distal and proximal motion members for coupling motion of the proximal motion member to the distal motion member for controlling the positioning of the tool; a control tube through which the instrument shaft and tool extend; the control tube including, along the length thereof, a curved section; the curved section of the control tube, upon rotation thereof, providing an additional degree of freedom by displacing the tool out of a plane defined by the curved section of the control tube.

2. The medical instrument of claim 1 wherein at least a portion of the length of the instrument shaft is flexible so as to enable the instrument shaft to pass through the curved section of the control tube.

3. The medical instrument of claim 2 including a ball member supported about the proximal motion member, the control tube having a distal end and a proximal end with the proximal end of the control tube fixedly attached to the ball member.

4. The medical instrument of claim 3 wherein the control tube is rigid and includes a straight section proximal to and contiguous with the curved section.

5. The medical instrument of claim 4 wherein the instrument shaft extends through the curved control tube so that the distal motion member and tool extend beyond the distal end of the curved control tube.

6. The medical instrument of claim 1 including a rotation knob at the control handle for rotating the instrument shaft and end effector about a longitudinal distal axis.

7. The medical instrument of claim 1 wherein both of the motion members are bendable members.

8. The medical instrument of claim 7 including a ball member supported about the proximal bendable member, the control tube having a distal end and a proximal end with the proximal end of the control tube fixedly attached to the ball member, and a locking mechanism disposed about the ball member.

9. The medical instrument of claim 8 wherein the locking mechanism includes a cinch ring that can be expanded and contracted.

10. The medical instrument of claim 1 wherein the control tube is rigid and includes a straight section proximal to and contiguous with the curved section, the straight and curved sections defining the plane.

11. The medical instrument of claim 1 including a ball member supported about the proximal bendable member, the control tube having a distal end and a proximal end with the proximal end of the control tube fixedly attached to the ball member, a locking ring disposed about the ball member and a pin and slot structure between the ball member and locking ring that permits oscillation of the ball member while preventing rotation of the ball member.

12. A medical instrument comprising: an instrument shaft having proximal and distal ends; a tool for performing a medical procedure; a control handle; a distal motion member for coupling the distal end of the instrument shaft to the tool; a proximal motion member for coupling the proximal end of the instrument shaft to the control handle; actuation means extending between the distal and proximal motion members for coupling motion of the proximal motion member to the distal motion member for controlling the positioning of the tool; a control tube through which the instrument shaft and tool extend; the control tube including, along the length thereof, a curved section; and a guide block having a slot therein for receiving the instrument shaft and control tube; the guide block disposed proximally of an anatomic port.

13. The medical instrument of claim 12 wherein the curved section of the control tube, upon rotation thereof, providing an additional degree of freedom by displacing the tool out of a plane defined by the curved section of the control tube.

14. The medical instrument of claim 12 wherein the control tube has at least three curved sections disposed therealong.

15. The medical instrument of claim 14 wherein two of the curved sections are proximal to the guide block and one of the curved sections is distal to the guide block.

16. The medical instrument of claim 12 including a pair of instruments and wherein the guide block has a corresponding pair of slots for receiving respective instrument shafts.

17. A medical instrument comprising: an instrument shaft having proximal and distal ends; a tool for performing a medical procedure; a control handle; a distal motion member for coupling the distal end of the instrument shaft to the tool; a proximal motion member for coupling the proximal end of the instrument shaft to the control handle; actuation means extending between the distal and proximal motion members for coupling motion of the proximal motion member to the distal motion member for controlling the positioning of the tool; a control tube through which the instrument shaft and tool extend; the control tube including, along the length thereof, at least one curved section; and an over tube having a passage therein for receiving the instrument shaft and control tube; the over tube disposed proximally of an anatomic port.

18. The medical instrument of claim 17 including at least one flexible articulation section along the length of the control tube.

19. The medical instrument of claim 18 wherein the curved section of the control tube is distal of the over tube and is rigid.

20. The medical instrument of claim 19 including a flexible articulation section on either side of the over tube and connected by cabling therebetween.

21. The medical instrument of claim 17 wherein the proximal motion member comprises a cable drive mechanism.

22. The medical instrument of claim 21 wherein the cable drive mechanism includes at least one motor, at least one pair of cables and a corresponding pair of followers driven by the motor.

23. The medical instrument of claim 22 including a threaded shaft for supporting the followers, driven from the motor and having opposed threads to drive the followers in opposite directions in controlling the cables.

24. The medical instrument of claim 23 including four cables and two motors mounted at the handle.

25. A cable drive mechanism supported in the handle of a surgical instrument for controlling a distal tool by controlling a bending of a distal bendable member that is coupled to the tool, said cable drive mechanism comprising, at least one motor, a pair of follower, a threaded shaft for supporting the pair of follower, a respective pair of control cables being connected to the followers at one end and to the distal bendable member at the other end thereof, the motor for driving the threaded shaft to, in turn, move the followers in either the same or opposite direction to control the tool.

26. The cable drive mechanism of claim 25 including a pair of motors and a pair of corresponding threaded shafts for each supporting a pair of followers.

27. The cable drive mechanism of claim 25 including as electrical control unit for controlling said motor.
